(19) United States
(12) Patent Application Publication
(10) Pub. No.: US 2024/0000883 A1
(43) Pub. Date: Jan. 4, 2024

(54) METHOD OF SENSITIZING CANCERS TO IMMUNOTHERAPY USING IMMUNOMODULATORY AGENTS

(71) Applicants: The Trustees of Columbia University in the City of New York, New York, NY (US); The Regents of the University of California, Oakland, CA (US)

(72) Inventors: Kazuki SUGAHARA, Fort Lee, NJ (US); Andrew M. LOWY, San Diego, CA (US)

(21) Appl. No.: 18/252,069

(22) PCT Filed: Dec. 1, 2021
(86) PCT No.: PCT/US21/72666

§ 371 Date: May 8, 2023

Related U.S. Application Data
(60) Provisional application No. 63/119,963, filed on Dec. 1, 2020.

(51) Int. Cl. A61K 38/12; A61P 35/00; C07K 16/28; A61K 45/06

(52) U.S. Cl. CPC A61K 38/12; A61P 35/00; C07K 16/2818; A61K 45/06; A61K 2039/505

(57) ABSTRACT

The invention described relates to the newly discovered ability of tumor internalizing arginylglycylaspartic acid (iRGD) peptides to alter the immune cell landscape in pancreatic ductal adenocarcinoma (PDAC) and other cancers. The iRGD peptides sensitize the cancer to immune checkpoint inhibitors, for example anti-PD-L1, anti-PD-L1, anti-PD-1, and anti-CTLA4 monoclonal antibodies to specifically deplete Tregs within the tumor, resulting in expansion of intratumoral CD8+ T cells (effector cells). This provides methods of treating cancers such as PDAC, preferably in synergistic combination with chemotherapy and immunotherapy, which leads to reduced tumor burden and prolonged survival.

### CROSS-REFERENCE TO RELATED APPLICATIONS

This application claims the benefit of U.S. provisional application Ser. No. 63/119,963, filed 1 Dec. 2020. The entire contents of this application is hereby incorporated by reference as if fully set forth herein.

### GOVERNMENT FUNDING SUPPORT

This invention was made with government support under grant no. CA167174 awarded by the National Institutes of Health. The government has certain rights in the invention.

### BACKGROUND

### 1. Field of the Invention

This invention relates to the field of medicine and oncology. In particular, the invention provides methods for the treatment of certain cancers using a combination of immunomodulatory compounds, including an immune checkpoint inhibitor and an iRGD peptide.

### 2. Background of the Invention

Cancer is among the leading causes of death worldwide. Despite recent advances in science, the impact of cancer immunotherapy on disease progression and overall survival has been limited to certain cancers such as melanoma and non-small cell lung cancer. Unfortunately, therefore, cancer immunotherapy is unable to elicit responses in a vast majority of cancers, including pancreatic cancer. About 92% of patients diagnosed with pancreatic cancer will die within five years of diagnosis. Cancers such as pancreatic ductal adenocarcinoma (PDAC) are almost completely refractory to all forms of chemo- and immuno-therapy. A highly immunosuppressive tumor microenvironment characterized by the presence of large numbers of regulatory T cells can drive resistance to immunotherapy. Therefore, there is a need for therapeutic agents to transform the tumor microenvironment to enhance immunotherapies for various cancers.

Immune checkpoints are a normal part of the immune system, which works to modulate immune responses so that they do not become so strong as to destroy healthy cells in the body. These immune checkpoints can engage when immune checkpoint proteins on the surface of T cells recognize and bind to partner proteins on other cells, resulting in an “off” signal for the T cells. When the other cells are tumor cells, this inhibition of the immune response can prevent the immune system from destroying the tumor.

Immunotherapy drugs called “immune checkpoint inhibitors” work by blocking checkpoint proteins from binding with their partner proteins on tumors. This prevents the “off” signal from being sent, allowing the T cells to kill cancer cells. Immune checkpoint inhibitors can act against a checkpoint protein called CTLA4 or a checkpoint protein called PD-1 or its partner protein PD-L1. Some tumors turn down the T cell response by producing lots of PD-L1.

A number of patients treated with immune checkpoint demonstrate tumor regression or prolonged stable disease, and some striking responses have been observed. However, overall, only a limited proportion of patients respond, and a significant number of patients experience adverse effects. Therefore, it would be of considerable benefit to be able to improve the number of patients who react positively to immunomodulatory therapy, and to provide ways to increase the likelihood that a patient will respond to immune checkpoint inhibitor therapy or to avoid or overcome lack of response to such therapy.

Because of the failure of immunotherapy to effectively treat many forms of cancer, and because many patients do not respond to immune checkpoint inhibitors, there is a great need in the art for methods of treating cancer with immunotherapies or immunomodulatory therapies.

### SUMMARY OF THE INVENTION

Cancer immunotherapy is ineffective in a vast majority of tumors, due to the immunosuppressive tumor microenvironment that prevents the infiltration and effector function of antitumor adaptive T cells. This application describes the immunomodulatory ability of tumor internalizing RGD peptides (iRGD) to sensitize a wide variety of refractory cancers to either or both of immunotherapy and chemotherapy. Therefore, this technology has the potential to greatly increase the efficacy of existing cancer immunotherapeutics and prevent tumor resistance. Previously, earlier work did not recognize that iRGD itself is immunomodulatory.

iRGD peptides can target and deplete immunosuppressive regulatory T cells in a tumor-specific manner Tumor infiltrating regulatory T cells (Tregs) are enriched in immunotherapy-refractory tumors such as pancreatic ductal adenocarcinoma (PDAC), contributing to their immunosuppressive tumor microenvironment. Treatments that cause a systemic depletion of Tregs are undesirable due to inflammatory, autoimmune side effects following non-specific eradication. Therefore, since iRGD receptors are only present in the tumor, using a peptide to specifically target these receptors enables effector CD8 T-cell expansion within the tumor, while preventing autoimmune toxicities that arise from systemic regulatory T cell depletion.

iRGD, a 9-amino acid cyclic peptide promotes tumor-specific cell and tissue penetration of linked drugs/proteins by binding to αv integrins. iRGD therapy sensitizes PDAC tumors to both chemotherapy and immune-checkpoint blockade, resulting in a significant reduction in tumor burden and prolonged survival in animal models. Notably, this technology also can be utilized in other peritoneal tumors due to the enrichment of iRGD receptors in tumor-infiltrating regulatory T cells in multiple tumor types. As such, iRGD peptides may significantly improve patient outcomes and overall survival in several immunotherapy-refractory cancers. In addition, this therapeutic agent synergizes with existing cancer therapeutics, leading to reduced tumor burden and improved survival in animal models of pancreatic cancer.

According to one embodiment, provided is a method comprising administering to the subject an iRGD peptide, or peptide variant thereof, or iRGD conjugate in combination with one or more immune checkpoint inhibitor. In a specific embodiment the iRGD peptide comprises the sequence defined in SEQ ID NO:3. In a further specific embodiment, the immune checkpoint inhibitor comprises a PD-1 inhibitor, a PD-L1 inhibitor, or a PD-L2 inhibitor or any combination thereof. The one or more immune checkpoint inhibitor may comprise 2, 3, or 4 immune checkpoint inhibitors. Examples of immune checkpoint inhibitors include, but are not limited to, ipilimumab, tremilimumab, nivolumab, pembrolizumab (lambrolizumab), pidilizumab, MPDL3280A, BMS-936559, MPDL3280A, MEDI4736, MSB0010718C, or any combination thereof. The method may further comprise administering to the subject an adjunct cancer therapy selected from the group consisting of surgery, radiation therapy, additional immunotherapy, and chemotherapy. The method embodiments described herein may treat a wide variety of cancers as is discussed further below.

### BRIEF SUMMARY OF THE DRAWINGS

FIG. 1A is a graph showing the percent survival of transgenic Kras-LSL^{GD12}, p53-LSL^{172H}, Pdx-1-cre (KPC) mice bearing de novo pancreatic ductal adenocarcinoma (PDAC) treated with gemcitabine (GEM).

FIG. 1B is a set of photographs of tumor collected from the mice in FIG. 1A, stained for CD8⁺ T cells. The cells were counted under a microscope using a randomly selected field of view, results of which are shown in FIG. 1C. In FIG. 1D, CD8⁺ T cells in the PDAC of the three most long-lived and the four most short-lived KPC mice were analyzed. Scale bars, 100 μm; *, p<0.05; ***, p<0.001.

FIG. 2A is a pair of photographs showing KPC organoids with elaborate folding and a lumen (arrowhead).

FIG. 2B is a set of graphs showing data on PD-L1 expression in luciferase-positive KPC (KPC-luc) organoids analyzed by flow cytometry.

FIG. 2C is a set of photographs of longitudinal luminescence imaging of orthotopic KPC-luc tumors in B6129SF1/J mice.

FIG. 2D is a set of images of KPC-luc PDAC and liver and lung metastases. H&E staining of the primary tumor is shown. Scale bar, 100 μm.

FIG. 2E, FIG. 2F, and FIG. 2G show results of flow cytometry of CD8⁺ T cells and Tregs (FIG. 2E), NPR-1⁺ Tregs (FIG. 2F), and αvβ3⁺ and αvβ5⁺ Tregs (FIG. 2G) in PDAC and spleen (Spl) of normal mice (NMs) and KPC-luc mice (PDAC Ms).

FIG. 2H is an image showing that intravenously injected FAM-iRGD (green) targets CD4⁺ (magenta) Foxp3⁺ (red) Tregs in KPC-luc PDAC (white arrowheads). Some iRGD-targeted Foxp3⁺ cells were CD4^{neg} (black arrowheads). Blue=DAPI. Scale bar, 50 μm.

FIG. 2I presents data on αvβ5 and NRP-1 expression in normal mouse spleen Tregs cultured alone or with KPC-luc cells.

FIG. 2J, FIG. 2K, FIG. 2L, and FIG. 2M present data for orthotopic KPC-luc mice treated with IV iRGD+GEM with or without anti-PD-L1 mAb (clone 10F.9G2) 3× a week for 2 weeks. The results show that iRGD+GEM significantly enhanced anti-PD-L1 therapy (FIG. 2J), NRP-1⁺ αvβ3 integrin⁺ total Tregs (FIG. 2K) and CD25^{high} Tregs (FIG. 2L, insets), and the proportion of CD8⁺ and CD4⁺ T cells (FIG. 2M), in the PDAC and spleen after iRGD+GEM+anti-PD-L1 mAb treatment. Statistics, ANOVA; n.s., not significant; ***, p<0.001.

FIG. 3A through FIG. 3B relates to αv integrin and NRP-1 expression in human PDAC Tregs. FIG. 3A shows expression of αvβ5 integrin in Tregs isolated from tumor (blue) and spleen (red) samples from a PDAC patient. Green is an isotype control. FIG. 3B is a pair of images showing αvβ5 integrin (green) in CD3⁺ (red) Foxp3⁺ (magenta) T cells (white arrowheads) and NRP-1 (green) in CD3⁺ T cells (yellow arrowheads) in human PDAC. Foxp3 was not stained in the right panel due to the incompatibility with NRP-1 staining DAPI not shown for better visualization of the other colors. Scale bars, 20 μm.

FIG. 4A through FIG. 4D are a set of graphs showing T cells in peritoneal tumors (PTs) in mice generated with ID8 mouse ovarian cancer cells: FIG. 4A, CD8⁺ (4%) and CD4⁺ (17%) T cells; FIG. 4B, CD25^{high} (32%) and CD25^{low} (58%) Tregs; FIG. 4C, αvβ3⁺ NRP-1⁺ Tregs (63%); FIG. 4D, αvβ5⁺ NRP-1⁺ Tregs (26%). The number of T cells was low since the PTs were small.

FIG. 5. Expression of αvβ5 integrin on Tregs and CTLs isolated from orthotopic PDAC (T) and spleen (S) of KPC-derived syngeneic tumor mice (T Ms) and the spleen of normal mice (N Ms) analyzed by flow cytometry. p**<0.01.

FIG. 6. Survival of CD4⁺ T cells in the presence or absence of KPC-derived PDAC cells. Splenic T cells from mice were cultured in the presence of KPC-derived PDAC cells to expand αvβ5 integrin⁺ Tregs. Survival was determined by counting the number of cells using a hemocytometer. *, p<0.01.

FIG. 7. iRGD binding to CD25⁺ CD4⁺ T cells (Tregs) and CD25^{neg} CD4⁺ T cells (non-Tregs) isolated from KPC-derived PDAC. The T cells were cultured in the presence of fluorescein (FAM)-labeled iRGD at 37° C. for 1 hrs. iRGD binding was determined by flow cytometry.

FIG. 8A and FIG. 8B. iRGD binding to CD25⁺ CD4⁺ T cells (Tregs) and CD25^{neg} CD4⁺ T cells (non-Tregs) produced in vitro. The Tregs and non-Tregs were produced by culturing mouse splenic T cells in the presence of CD3/CD28 beads and KPC-derived PDAC cells. FIG. 8A, FAM-iRGD binding to the Tregs was determined by flow cytometry. FIG. 8B, anti-αvβ5 integrin Abs inhibited FAM-iRGD binding to the Tregs.

FIG. 9A and FIG. 9B. The effect of iRGD monotherapy on Tregs and the CTL/Treg ratio in the PDAC tissue and spleen. Mice bearing orthotopic PDAC were treated with systemic iRGD or PBS for 2 weeks. FIG. 9A, Time-dependent changes in the proportion of Tregs and FIG. 9B CTL/Treg ratio in the PDAC tissue.

FIG. 10A, αvβ5 integrin⁺ and FIG. 10B NRP-1⁺ Tregs in the PDAC after iRGD monotherapy.

FIG. 11A, Time-dependent changes in the proportion of Tregs and FIG. 11B, CTL/Treg ratio in the spleen. *, p<0.05; n.s., not significant.

FIG. 12A and FIG. 12B. KPC-derived PDAC mice were treated with iRGD±anti-PD-L1 mAb (A; n=4-6) or iRGD+Gem±anti-PD-L1 mAb (B; n=4) 3× a week for 2 weeks. FIG. 12B. Flow cytometry data of CD4⁺ CD25⁺ Tregs and CD8⁺ T cells in the tumor and spleen after iRGD+Gem+anti-PD-L1 mAb therapy are shown. Tregs halved and CTLs doubled in the PDAC but not in the spleen. n.s., not significant; *, p<0.05; **, p<0.01.

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about,” means plus or minus 20 percent of the recited value, so that, for example, “about 0.125” means 0.125±0.025, and “about 1.0” means 1.0±0.2.

As used herein, the term “iRGD” or “iRGD peptide” refers to a 9-amino acid cyclic peptide having sequence (sequence: CRGDKGPDC; SEQ ID NO:2) or a variant thereof. In certain specific examples, variants of iRGD include the following CRGD(R/K/H)G(P/V)(D/E/H)C (SEQ ID NO:3), wherein the parentheses set forth amino acid options at that position. Other iRGD variants are disclosed in US Pat. Pub. No, 20090246133, which is incorporated herein in its entirety. Reference to iRGD, iRGD peptide or peptide includes peptide variants unless stated otherwise.

As used herein, the terms “treatment,” “treating,” and the like, refer to obtaining a desired pharmacologic and/or physiologic effect through administering compound(s) or composition(s). “Treatment,” includes: preventing, partially preventing, reversing, alleviating, reducing the likelihood of, or inhibiting the condition or disease (or symptom thereof) from occurring in a subject. The subject can include those diagnosed with a tumor or cancer, a pre-cancer, or who are predisposed to the condition or disease but has not yet been diagnosed as having it; (b) inhibiting the condition or disease or symptom thereof, such as, arresting its development; and (c) relieving, alleviating or ameliorating the condition or disease or symptom thereof, such as, for example, causing regression of the condition or disease or symptom thereof. Treatment can include administering one or more agents, performing a procedure such as surgery or applying radiation and the like, or both.

As used herein, the term “administering” and its cognates refer to introducing an agent to a subject, and can be performed using any of the various methods or delivery systems for administering agents or pharmaceutical compositions, and any route suitable for the composition and the subject, as known to those skilled in the art. Modes of administering include, but are not limited to oral administration, intravenous, subcutaneous, intramuscular or intraperitoneal injections, or local administration directly into or onto a target tissue (such as the pancreas, brain, or a tumor). Administration by any route or method that delivers a therapeutically effective amount of the drug or composition to the cells or tissue to which it is targeted is suitable for use with the invention.

As used herein, the term “combination,” with respect to administration of more than one active agent to a subject, i.e., combination therapy, refers to administration simultaneously or at different times. The one or more agents can be delivered in two or several pharmaceutical compositions that contain one active agent each, or using pharmaceutical compositions that each contain one or more active agent(s). The different pharmaceutical compositions can be formulated for the same or different routes of administration. The administration of the separate pharmaceutical compositions can be accomplished at the same time, in quick succession, or separated in time by minutes, hours, days, or weeks. Combination treatment with an immune checkpoint inhibitor and iRGD may be presumed to be the case if an immune checkpoint inhibitor and a complement inhibitor are prescribed or administered to a subject suffering from cancer by or under direction of the same health care professional. A combination pharmaceutical composition contains more than one active agent and a pharmaceutically acceptable carrier.

As used herein, the terms “subject,” “individual,” “host,” and “patient,” are used interchangeably to refer to humans or any non-human mammal, and can include mammalian farm animals, mammalian sport animals, mammalian companion animals, simians, non-human primates, felines, canines, equines, rodents, lagomorphs, bovines, porcines, ovines, caprines. A suitable subject for the invention preferably is a human that is suspected of having, has been diagnosed as having, or is at risk of developing a hyperproliferative disease. Conditions amenable to treatment by the invention which define an appropriate subject or patient will be discerned easily by the person of skill in the art based on the disclosures herein. A “subject in need” is a subject that is at risk of developing cancer, or who manifests any characteristics or symptoms of cancer, or who has been diagnosed with cancer.

As used herein, the term “cancer”, also referred to as a tumor or a malignant tumor, refers to any of a group of diseases involving abnormal cell proliferation (hyperproliferation) with the potential to invade locally and/or spread to other parts of the body (metastasize). The term “cancer” is generally used interchangeably with “tumor” herein (unless a tumor is specifically referred to as a “benign” tumor, which is an abnormal mass of cells that lacks the ability to invade neighboring tissue or metastasize), and encompasses malignant solid tumors (e.g., carcinomas, sarcomas) and malignant growths in which there may be no detectable solid tumor mass (e.g., certain hematologic malignancies). In particular, cancers that are susceptible to immune checkpoint inhibitors are contemplated for use with the methods according to the invention, however immune checkpoint inhibitor-resistant cancers also can be treated according to embodiments of the invention. The term “cancer” can refer to a primary or metastatic tumor, and includes cancers that are unresectable cancer, and cancers of any stage, including stage III cancer and/or stage IV cancer.

As used herein, the term “antibody” refers to an immunoglobulin and encompasses full size antibodies and antibody fragments comprising an antigen binding site. Antibodies useful in certain embodiments of the invention may originate from or be derived from a mammal, e g, a human, non-human primate, rodent (e.g., mouse, rat), rabbit, goat, bovine, equine, ovine, camelid, or from a bird (e.g., chicken), and may be of any of the various antibody isotypes, e.g., the mammalian isotypes: IgG (e.g., of the IgG1, IgG2, IgG3, or IgG4 subclass), IgM, IgA, IgD, and IgE or isotypes that are not found in mammals, e.g., IgY (found in birds) or IgW (found in sharks).

An antibody fragment (Fab) may be, for example, a Fab′, F(ab′)₂, scFv (single-chain variable), single domain antibody (e.g., a VHH), or other fragment that retains or contains an antigen binding site. See, e.g., Allen, T., Nature Reviews Cancer, Vol. 2, 750-765, 2002, and references therein for disclosures relating to antibody fragments. The contents of this reference are hereby incorporated by reference. Antibodies known in the art as diabodies, minibodies, or nanobodies can be used in various embodiments. Bispecific or multispecific antibodies may be used in various embodiments. The heavy and light chain of IgG immunoglobulins (e.g., rodent or human IgGs) contain four framework regions (FR1 through FR4) separated respectively by three complementarity determining regions (CDR1 through CDR3). The CDRs, particularly the CDR3 regions and especially the heavy chain CDR3, are largely responsible for antibody specificity.

An antibody may be a chimeric antibody in which, for example, a variable domain of non-human origin, e.g., of rodent (e.g., murine) or non-human primate origin) is fused to a constant domain of human origin, or a “humanized” antibody in which some or all of the complementarity-determining region (CDR) amino acids that constitute an antigen binding site (sometimes along with one or more framework amino acids or regions) are “grafted” from a rodent antibody (e.g., murine antibody) or phage display antibody to a human antibody, thus retaining the specificity of the rodent or phage display antibody. Thus, humanized antibodies may be recombinant proteins in which only the antibody complementarity-determining regions are of non-human origin. Alterations to antibody sequence that are involved in the humanization process are generally carried out through techniques at the nucleic acid level, e.g., standard recombinant nucleic acid techniques. In some embodiments only the specificity determining residues (SDRs), the CDR residues that are most crucial in the antibody-ligand interaction, are grafted. The SDRs may be identified, e.g., through use of a database of the three-dimensional structures of the antigen-antibody complexes of known structures or by mutational analysis of the antibody-combining site. In some embodiments an approach is used that involves retention of more CDR residues, namely grafting of so-called “abbreviated” CDRs, the stretches of CDR residues that include all the SDRs. See, e.g., Kashmiri, S V, Methods. 36(1):25-34 (2005), for further discussion of SDR grafting and Almagro J C, Fransson J. Humanization of antibodies. Front Biosci. 13:1619-33 (2008) for review of various methods of obtaining humanized antibodies. These references are incorporated by reference herein. “Originate from or derived from” refers to the original source of the genetic information specifying an antibody sequence or a portion thereof, which may be different from the species in which an antibody is initially synthesized. For example, “human” domains may be generated in rodents (e.g., mice) whose genome incorporates human immunoglobulin genes or may be generated using phage display. See, e.g., Vaughan, et al, (1998), Nature Biotechnology, 16: 535-539, e.g., for discussion of methods that may be used to generate a fully human antibody. This reference is incorporated by reference.

The amino acid sequences of the variable regions of such antibodies are sequences that, while derived from and related to the germline sequences encoding variable domains (V_{H} and/or V_{L} domains) of a particular species (e.g., human), may not naturally exist within that species' antibody germline repertoire in vivo. For example, the human immunoglobulin genes may have been subjected to in vitro mutagenesis (or, when an animal transgenic for human immunoglobulin gene sequences is used, in vivo somatic mutagenesis). Antibodies suitable for use with the invention may be polyclonal or monoclonal, though for purposes of the present invention monoclonal antibodies are generally preferred as therapeutic agents. Antibodies can be glycosylated or non-glycosylated.

Methods for generating antibodies that specifically bind to virtually any molecule of interest are known in the art. For example, monoclonal or polyclonal antibodies can be purified from natural sources, e.g., from blood or ascites fluid of an animal that produces the antibody (e.g., following immunization with the molecule or an antigenic fragment thereof) or can be produced recombinantly, in cell culture and, e.g., purified from culture medium. Affinity purification may be used, e.g., protein A/G affinity purification and/or affinity purification using the antigen as an affinity reagent.

Suitable antibodies can be identified using phage display and related techniques. See, e.g., Kaser, M. and Howard, G., “Making and Using Antibodies: A Practical Handbook” and Sidhu, S., “Phage Display in Biotechnology and Drug Discovery”, CRC Press, Taylor and Francis Group, 2005, for further information. This reference is incorporated by reference.

Methods for generating antibody fragments are well known. For example, F(ab′)₂ fragments can be generated, for example, through the use of an Immunopure F(ab′)₂ Preparation Kit (Pierce™) in which the antibodies are digested using immobilized pepsin and purified over an immobilized Protein A column. The digestion conditions (such as temperature and duration) may be optimized by one of ordinary skill in the art to obtain a good yield of F(ab′)₂. The yield of F(ab′)₂ resulting from the digestion can be monitored by standard protein gel electrophoresis. F(ab′) can be obtained by papain digestion of antibodies, or by reducing the S—S bond in the F(ab′)₂. A “single-chain Fv” or “scFv” antibody fragment comprises the V_{H} and V_{L} domains of an antibody, wherein these domains are present in a single polypeptide chain. Typically, an scFv antibody further comprises a polypeptide linker between the V_{H} and V_{L} domains, although other linkers could be used to connect the domains in certain embodiments.

As used herein, the term “monoclonal antibody” (MAb) or “monoclonal antibody composition” refers to a population of antibody molecules that contain only one molecular species of antibody molecule consisting of a unique light chain gene product and a unique heavy chain gene product. In particular, the complementarily determining regions (CDRs) of the monoclonal antibody are identical in all the molecules of the population.

As used herein, the term “immune system cell” refers to any of a variety of cells that play a role in the immune response Immune system cells include lymphocytes (T cells, B cells, natural killer (NK) cells), dendritic cells, monocytes, macrophages, eosinophils, mast cells, basophils, and neutrophils. T cells comprise a number of different functional classes that play different roles in the immune response. Different functional classes may be distinguished based on cell surface markers and other properties. Most T cells express an αβ T cell receptor (TCR) through which the cell is able to recognize a specific antigen in the context of an appropriate major histocompatibility complex (MHC) molecule, though a minor subset expresses the γδ TCR.

Cytotoxic T cells (CTLs) are typically positive for the cell surface marker CD8, which serves as a co-receptor for the TCR in recognition of MHC Class I molecules on the surface of target cells during antigen-specific T cell activation and/or responses. CTLs and NK cells play important roles by eliminating infected host cells and tumor cells through a variety of mechanisms including the release of cytotoxic substances.

Helper T cells are typically positive for the cell surface marker CD4, which serves as a co-receptor for the TCR in recognition of MHC Class II molecules on the surface of APCs during antigen-specific T cell activation. Helper T cells promote the activity of other immune system cells (i.e., provide “help”) by, among other things, releasing cytokines that have a variety of effects such as enhancing survival, proliferation, and/or differentiation.

Natural killer cells have the ability to recognize and kill (e.g., by causing lysis or apoptosis) cancerous, stressed, or infected cells without requiring antigen-specific activation by presentation of antigen in the context of MHC. Instead, their activation is regulated by a balance of the activity of activating receptors and inhibitory receptors and cytokines. NK cells typically lack cell surface receptors that are highly specific for a particular antigen and are able to react rapidly without prior exposure to the antigen.

As used herein, “effector cells” refers to the activated immune system cells that defend the body in an immune response. Effector T cells include cytotoxic T cells and helper T cells, which carry out cell-mediated responses. Effector B cells are called plasma cells and secrete antibodies. Effector cells also include effector NK cells.

An antigen-presenting cell (APC) is a cell that can process and display antigens in association with major histocompatibility complex (MHC) molecules on its surface. T cells can recognize these complexes using their T cell receptors (TCRs). APCs also can display other molecules (costimulatory proteins) that are required for activating naive T cells. APCs that express MHC class II molecules include dendritic cells, macrophages, and B cells and may be referred to as professional APCs.

Dendritic cells (DCs) are white blood cells that occur in most tissues of the body, particularly epithelial tissues. DCs serve as a link between peripheral tissues and lymphoid organs. Immature DCs sample the surrounding environment and take up antigenic substances such as pathogen components or tumor antigens. They undergo maturation and migrate to lymph nodes or spleen, where they display fragments of processed antigens at their cell surface using MHC Class II (MHCII) complexes. As part of the maturation process, DCs upregulate cell-surface molecules that act as co-stimulators in T cell activation, such as CD80 (B7-1), CD86 (B7-2), and/or CD40. DCs activate helper T cells by presenting them with antigens in the context of MHCII complexes, together with non-antigen specific co-stimulators. DCs and various other APCs have the capacity to activate cytotoxic T cells and B cells through presentation of MHC Class I (MHCI)-peptide complexes (cross-presentation) and costimulators.

As used herein, the term “regulatory T cells (Tregs, suppressor T cells)” refers to a subpopulation of CD4+ T cells which modulate the immune system, maintain tolerance to self-antigens, and abrogate autoimmune disease. These cells generally suppress or downregulate induction and proliferation of effector T cells and can be identified based on a cell surface marker expression pattern of CD4+ CD25+ CD127^{lo}. Tregs also are characterized by expression of CTLA4 and GITR. Tregs can suppress the activity of other immune system cell subsets by a variety of mechanisms such as secretion of immunosuppressive cytokines and via cell-cell contact. They can inhibit immune responses at multiple steps, e.g., at the induction of activation (e.g., by inhibiting the ability of APCs to stimulate T cells) and during effector phases. Tregs are often found in tumors, and increased numbers of Tregs has been associated with a worse prognosis in various cancer types. Where it is intended herein to refer to a T cell that is a Treg, the T cell will be identified as such. Thus, unless expressly indicated a T cell, as used herein, is not a Treg cell.

As used herein, the term “adjunct cancer therapy” refers to a therapy, such as surgery, chemotherapy, radiotherapy, thermotherapy, and laser therapy, that can provide a beneficial effect when administered in conjunction with administration of iRGD in optional combination with an immune checkpoint inhibitor. The term “anti-cancer agent” refers to conventional chemotherapy, a molecularly targeted anticancer agent, a cancer vaccine, a second immunostimulatory agent, cell-based immunotherapy, or a combination thereof to the subject.

As used herein, an “adjunct cancer therapeutic agent” refers to an agent, compound, or composition that possesses selectively cytotoxic or cytostatic effects on cancer cells compared to normal cells. Adjunct cancer therapeutic agents can be co-administered with an iRGD, and/or an immune checkpoint inhibitor. A non-limiting list of examples of selected adjunct cancer therapeutic agents is provided in Table 1, below.

As used herein, a “peptide” is a sequence of two or more amino acids up to about 100 amino acids. A “variant” of a particular peptide has one or more alterations (e.g., additions, substitutions, and/or deletions, which may be referred to collectively as “mutations”) with respect to the original peptide sequence. Thus, a variant can be shorter or longer than the original peptide of which it is a variant. Conservative substitutions are preferred when substitutions are made in a peptide. Conservative substitutions are those where an amino acid is replaced with a different amino acid of the same type, such as glutamic acid for aspartic acid or alanine for glycine and the like. Persons of skill are aware of such substitutions.

The term “variant” also encompasses “fragments.” A “fragment” is a continuous portion of a polypeptide that is shorter than the original peptide. In certain embodiments of the invention a variant peptide has significant sequence identity to the original polypeptide over a continuous portion of the variant that comprises at least 70%, at least 80%, at least 90%, at least 95%, or more, of the length of the peptide. Peptides can include non-traditional amino acids or D-amino acids as well, or terminal additions or modification such as C-terminal amides, and the like. An amino acid “difference” refers to a substitution, insertion, or deletion of an amino acid. In certain embodiments, peptide variants also encompass peptidomimetics of a peptide or peptide mimics.

The term “peptidomimetic,” as used herein, means a peptide-like molecule that has the activity of the peptide upon which it is structurally based. Such peptidomimetics include chemically modified peptides, peptide-like molecules containing non-naturally occurring amino acids, and peptoids and have an activity such as that from which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861).

As used herein the term “immune checkpoint protein” refers to a protein or receptor that functions in an immune checkpoint pathway. Examples of immune checkpoint proteins include inhibitory receptors through which an immune checkpoint pathway is initiated, and their ligands. Examples of immune checkpoint pathways include the cytotoxic T-lymphocyte associated antigen 4 (CTLA4) pathway and the programmed cell death 1 (PD1) pathway, both of which are further discussed below. The term “immune checkpoint molecule” encompasses immune checkpoint proteins as well as small molecules such as adenosine that play a role in immune checkpoint pathways.

As used herein, the term “immune checkpoint inhibitor” refers to a class of agents that activate the immune system to attack tumors by blocking or reducing the activity of immune checkpoint molecules such as CTLA4, PD-1, PD-L1, and the like, discussed below.

As used herein, the term “effective amount” of an active agent, e.g., an immune checkpoint inhibitor or an iRGD peptide, refers to an amount of the active agent sufficient to elicit one or more biological effect(s) of interest in, for example, a subject to whom the active agent (or composition) is administered. In some embodiments the biological effect of an active agent is enhancement of the efficacy of a second agent.

As will be appreciated by those of ordinary skill in the art, the absolute amount of a particular agent that is effective may vary depending on such factors as the biological endpoint, the particular active agent, the target tissue, etc. An effective amount of an agent or composition generally is an amount sufficient to achieve one or more of the following in a cancer patient: a complete response (remission), a partial response, achievement of stable disease as determined by objective criteria, an improvement in symptoms, an increase in the length of progression-free survival, or an increase in overall survival. An effective amount can be an amount that results in killing of tumor cells, directly or indirectly or that stops growth of the tumor cells. Those of ordinary skill in the art will further understand that an “effective amount” may be administered in a single dose, or may be achieved by administration of multiple doses over a period of time. An effective amount of a pharmaceutical composition that contains an effective amount of one or more agents is an amount of each agent such that the overall composition is effective.

In some embodiments, an effective amount of an agent or composition can be an amount that suppresses (e g, eliminates) replication of a pathogen in a subject suffering from an infection, renders a subject free of the infectious agent, renders the subject non-infectious, results in an improvement in symptoms of infection, decreases mortality due to the infection, and/or an increases overall survival.

### 2. Overview

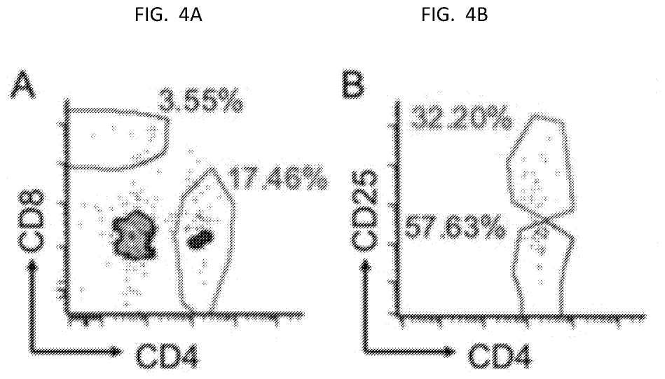

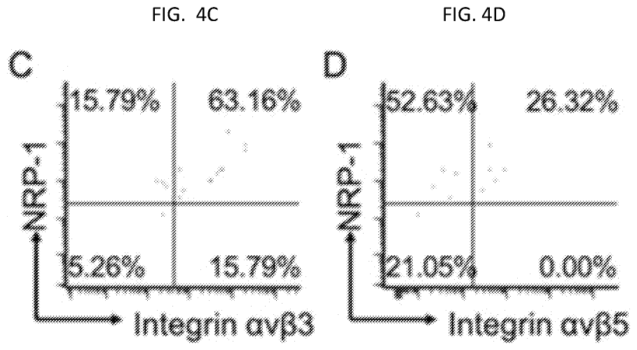

The invention is based on the discovery that iRGD tumor penetrating peptide possesses immunomodulatory effects that allow it to be used for treatment of tumors in conjunction with immune checkpoint inhibitors. The data presented herein show a potentiating, synergistic effect on cancer chemotherapeutic agents when iRGD is co-administered with immune checkpoint inhibitors. This technology can also be applied to intraperitoneal chemotherapy methods because iRGD (and co-administered drugs) target various peritoneal tumors when administered intraperitoneally indicating that it will likely sensitize peritoneal metastases of various tumors, such as ovarian cancer (see FIG. 4), to immunotherapy.

The concept of tumor-specific immunotherapy is becoming increasingly important because non-specific eradication of Tregs can cause inflammatory side effects. Therefore, the invention makes certain tumor-specific immunotherapies feasible. iRGD does not have to be conjugated to any of the co-administered agents to be effective. The method allows immunomodification with enhanced immunotherapy by simple co-administration. That being said, iRGD conjugates may be produced and used in combination with immune checkpoint inhibitors. Specific examples of iRGD conjugates include iRGD conjugated with an adjunct cancer therapeutic agent.

### 3. Summary of the Results

iRGD can modulate the immune landscape in pancreatic duct adenocarcinoma (PDAC), sensitizing the cancer to immune checkpoint inhibitors (i.e. anti-PD-L1, anti-PD-1, and anti-CTLA4 mAbs).

iRGD specifically depletes Tregs within the tumor.

iRGD results in expansion of intratumoral CD8+ T cells (effector cells) in PDAC.

iRGD enables synergy with chemotherapy and immunotherapy leading to reduced tumor burden and prolonged survival in a PDAC mouse model.

iRGD does not have to be conjugated to anti-cancer drugs or immunotherapeutics.

iRGD enhances immunotherapy via co-administration with anti-cancer drugs or other immunotherapeutics.

iRGD can be applied to intraperitoneal chemotherapy.

iRGD can improve the effectiveness of cancer immunotherapies, since αv integrin NRP-1⁺ Tregs are expressed exclusively within tumors in multiple cancers.

### 4. Embodiments of the Invention

A. Immunotherapies

Immunotherapy is a type of cancer treatment that assists the immune system in fighting cancer. The therapy stimulates the immune system to find and attack cancer cells rather than directly killing the cancer like traditional cancer chemotherapy drugs. Most cancer immunotherapy exploits the fact that tumor cells often have specific tumor antigens on their surface that can be specifically recognized and targeted by immune molecules such as antibodies or modified antibodies Immunotherapies according to this invention

B. Immune Checkpoints

An important function of the immune system is its ability to tell between normal cells in the body and those it sees as “foreign.” This lets the immune system attack the foreign cells while leaving the normal cells alone. To do this, it uses “checkpoints.” Immune checkpoints are molecules on certain immune cells that need to be activated (or inactivated) to start an immune response. In summary, immune checkpoints are immune system regulators that are crucial for self tolerance, which prevents the immune system from attacking normal cells. However, some tumors can protect themselves from attack by the immune system by manipulating this system. Drugs that target these checkpoints hold a lot of promise as cancer treatments. These drugs are called checkpoint inhibitors.

Immune checkpoint molecules can be stimulatory (e.g., members of the tumor necrosis factor receptor superfamily such as CD27, CD40, OS40, GITR, and CD137) or inhibitory (e.g., A2AR, B7-H3, B7-H4, BTLA, CTLA4, IDO, KIR, LAG3, NOX2, PD-1, TIM-3, VISTA, and SIGLEC7).

PD-1 is a checkpoint protein on T cells. It normally acts as a type of “off switch” that helps keep the T cells from attacking other cells in the body when it binds its ligand, which is present on some normal and cancer cells. When PD-1 binds to PD-L1, it sends the message for the T cell not to attack the other cell. Some cancer cells have large amounts of PD-L1, which helps them hide from an immune attack. The binding of PD-L1 to PD-1, for example, keeps T cells from killing tumor cells in the body. Blocking this binding with an immune checkpoint inhibitor allows effector T cells to attack and kill turner cells.

C. Immune Checkpoint Inhibitors

Immune checkpoint inhibitors are molecules (drugs) that inhibit or block inhibition of the immune system, such as by blocking inhibitory checkpoint proteins. Examples of checkpoint proteins include CTLA4, and/or PD-1, and/or PD-L1 and/or PD-L2. Pembrolizumab (lambrolizumb; Keytruda), Nivolumab (Opdivo), Atezolizumab (Tecentriq), Avelumab (Bevancio), cemiplimab (Libtayo), and Dumalumab (Imfinizi) are FDA-approved drugs that inhibit PD-1/PD-L1, and are contemplated for use with the invention. Additional immune checkpoint inhibitors include MEDI0680, MPDL3280A, AMP-224, BMS-936559, MPDL3280A, MEDI4736, MSB0010718C, for example. The synergistic effects of with immune checkpoint inhibitors should apply to any of the foregoing immune checkpoint inhibitors, or newly developed immune checkpoint inhibitors targeting the aforementioned inhibitory checkpoint proteins or other inhibitory checkpoint proteins to be elucidated. (See Freeman et al, JCI 130:1405-1416, 2020)

Preferably, the immune checkpoint inhibitor comprises an antibody, aptamer, non-antibody engineered binding protein, dominant negative protein, or other specific binding agent that binds to an inhibitory immune checkpoint molecule, e.g., CTLA4 or PD1.

Other PD1 pathway inhibitors also can include RNAi agents or antisense oligonucleotides that inhibit expression of PD1.

Preferred immune checkpoint inhibitors are monoclonal antibodies that target either PD-1 or PD-L1 to block this binding and boost the immune response against cells. Preferred examples of drugs that target and antagonize or block PD-1 include: pembrolizumab (Keytruda); nivoluniab (Opdivo); and cemiplirnab (Libtayo). Preferred examples of drugs that target and block or antagonize PD-L1 include: atezolizumab (Tecentriq); avelumab (Bavencio); and durvalumab (Imfinzi). These drugs can be helpful treating several types of cancer.

All combinations of any genus, subgenus, or species of immune checkpoint inhibitor and any genus, subgenus, or species of complement inhibitor, compositions comprising any such combination, and use of any such combination in any method described herein, are to be considered expressly disclosed herein. Any antibody or other specific binder that can block or inhibit CTL-4, and/or PD-1 and/or PD-L1 and/or PD-L2 can be used with the inventive methods, such as nanoparticles, engineered cells, any engineered binding protein, soluble receptor, aptamer, peptide or small molecule that binds to an immune checkpoint protein and preferably antagonizes or blocks an inhibitory immune checkpoint molecule. Combinations or mixtures of any of such immune checkpoint inhibitors are suitable for use with the invention.

In certain embodiments, the immune checkpoint inhibitor comprises ipilimumab and/or tremelimumab, which inhibit the CTLA4 pathway. In some embodiments of the invention, the immune checkpoint inhibitor inhibits a killer-like immunoglobulin receptor (KIR) pathway. For example, in some embodiments the immune checkpoint inhibitor binds to a KIR or KIR ligand. In some embodiments of the invention, the immune checkpoint inhibitor inhibits an immune checkpoint pathway involving LAG3, TIM3, BTLA, A2AR, or A2BR.

PD1 has two known ligands, PD1 ligand 1 (PD-L1; also known as B7-H1 and CD274) and PD-L2 (also known as B7-DC). The PD-1 pathway limits the activity of T cells in peripheral tissues at the time of an inflammatory response to infection and in order to limit autoimmunity. PD1 is a member of the CD28/CTLA4 family that is expressed on activated T cells. Binding of PD1 by its ligands mediates an inhibitory signal that results in reduced cytokine production, and reduced T cell survival. PD1 expression is induced when T cells become activated. When engaged by one of its ligands, PD1 inhibits kinases that are involved in T cell activation.

Like CTLA4, PD1 is highly expressed on Treg cells, and its activation can enhance their proliferation and/or suppressive activity in the presence of a PD1 ligand, which further suppresses immune function. Since many tumors are highly infiltrated with Treg cells, blockade of the PD1 pathway increases antitumor immune responses by decreasing the number and/or suppressive activity of Treg cells.

A PD1 inhibitor is an agent that inhibits the activity of PD1 or its natural ligand(s) with the effect that PD1's ability to suppress immune responses is reduced. A PD1 pathway inhibitor encompasses any agent that impairs the ability of PD1 to limit T cell activity or enhance Treg proliferation and/or suppressor functions. In some embodiments a PD1 inhibitor specifically binds to PD1 and inhibits its activation or activity. In some embodiments a PD1 inhibitor specifically binds to PD1 and blocks interaction of PD1 with its ligands.

In some embodiments a PD1 inhibitor (or a PD-L1 inhibitor or PD-L2 inhibitor) binds with a Kd of about 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, e.g., between 10⁻¹³ M and 10⁻⁶ M, or within any range having any two of the afore-mentioned values as endpoints. In some embodiments a PD1 inhibitor (or a PD-L1 inhibitor or PD-L2 inhibitor) binds with a Kd of no more than 10-fold that of nivolumab, up to 10-fold lower, or up to 100-fold lower than that of nivolumab when compared using the same assay.

In some embodiments, the IC50 values for inhibition by a PD1 inhibitor of PD1 binding to its ligands is no more than 10-fold greater, up to 10-fold lower, or up to 100-fold lower than that of nivolumab-mediated inhibition of PD1 binding to its ligands, when compared using the same assay.

CTLA4 is expressed on T cells, and its principal function is to regulate the extent of the early stages of T cell activation. In general, activation of T cells typically occurs through engagement of the T cell receptor (TCR) and a costimulatory molecule on the T cell. Binding of the T cell receptor to a processed form of its cognate antigen (an antigen to which an antigen receptor binds) presented by major histocompatibility complex (MHC) molecules on an antigen presenting cell provides a first signal for activation. The second signal comes from co-stimulation, in which surface molecules on the antigen presenting cell bind to co-stimulatory receptors on T cells and activate intracellular signaling pathways. CD28 is the most important co-stimulatory receptor for T cell activation and is expressed constitutively by naive T cells (cells that have not encountered cognate antigen). In the absence of co-stimulation, T-cell receptor signaling alone can result in anergy.

CD28 and CTLA4 display a different pattern of expression on T-cells: while CD28 is constitutively expressed on the surface of T-cells, CTLA4 is detectable at low levels in naive T-cells and more strongly upon T-cell activation. CTLA4 has the same ligands as does CD28, but the affinity of CTLA4 is about 10-fold higher than that of CD28. CTLA4 expression on T cells may counteract the activity of CD28 by competing for ligand binding, may actively deliver inhibitory signals to the T cell, or both. Through these and/or other mechanisms, CTLA4 inhibits T cell activation, thus reducing immune responses and anti-tumor immunity. CTLA4 is also expressed by Tregs and promotes their immune suppressive function, further contributing to impairing the immune response to the tumor.

A CTLA4 inhibitor is an agent that inhibits the activity of CTLA4 with the effect that the biological activity of CTLA4 is inhibited or reduced, e.g., that impairs the ability of CTLA4 to cause inhibition of T cell activation or impairs the ability of CTLA4 to enhance Treg proliferation and/or suppressor function. A preferred CTLA4 inhibitor is an agent that specifically binds to CTLA4 and inhibits its activation or activity.

U.S. Pat. Nos. 5,811,097; 5,855,887; 5,977,318; 6,051,227; 6,682,736; 6,207,156; 6,984,720; 7,109,003; 7,132,281; and 7,605,238, United States Patent Publication Nos. US2002-0039581, US2002-086014, US2004-0202650, US 2005-0201994, US2006-0165706, US 2011-0081354, US 2012-0148597, US2013-0011405, US2013-0136749, US2014-0105914, and US2014-0099325, international Patent Publication Nos. WO 2001/014424, WO 01/14424, WO 00/37504, WO 98/42752, and WO 2004/035607, and European Patent No. EP1212422B1 describe antibodies that bind to CTLA4 and are incorporated by reference for these disclosures. United States Patent Publication Nos. US2003-0054360 and US2006-0246123 disclose anti-CTLA4 aptamers that may be used in methods and compositions described herein.

In some embodiments of the invention a subject is treated with two or more immune checkpoint inhibitors in combination (administered together in the same bifunctional or multifunctional composition, or in separate compositions to be administered together or separately). The two or more immune checkpoint inhibitors can be provided or administered as part of a bifunctional or multifunctional agent or compound. For example, a bispecific, trispecific, or tetraspecific antibody (or other binding agent) capable of binding to two, three, or four distinct immune checkpoint molecules can be used.

The two or more immune checkpoint inhibitors can inhibit the same or different immune checkpoint pathways. For example, in some embodiments a first immune checkpoint inhibitor inhibits the PD1 pathway and a second or third immune checkpoint inhibitor inhibits the CTLA4 pathway. For example, any combination of two or more of ipilimumab, nivolumab, pembrolizumab, tremilimumab, pidilizumab, MEDI0680, BMS-936559, MPDL3280A, MEDI4736, MSB0010718C, or SB0010718C can be used in the same treatment method.

For example, in some embodiments a first immune checkpoint inhibitor agent inhibits PD1 or CTLA4 and a second agent comprises a TIM3 inhibitor, BTLA pathway inhibitor, KIR inhibitor, LAG3 inhibitor, or adenosine pathway inhibitor. In certain embodiments the method involves both a PD1 inhibitor and a CTLA4 inhibitor with a further TIM3 inhibitor, BTLA pathway inhibitor, KIR inhibitor, LAG3 inhibitor, IDO inhibitor, or adenosine pathway inhibitor. It is contemplated that the combination of immune checkpoint inhibitors comprises no more than 2, 3, 4, or 5 immune checkpoint inhibitors.

D. RGD

RGD tripeptide (RGD; SEQ ID NO:1) was originally identified as the amino acid sequence within the extracellular matrix protein fibronectin (the binding motif) that mediates cell adhesion/attachment. It also acts as an inhibitor of integrin-ligand interaction and can reduce apoptosis in the absence of signals and integrin-mediated cell clustering. The RGD motif has also been identified in other extracellular matrix proteins, including vitronectin and laminin.

E. iRGD Peptides and Peptidomimetics

(E.1) The iRGD peptide has been previously described in the art as a 9-amino acid cyclic peptide (CRGDKGPDC; SEQ ID NO:2) and a molecular mimicry agent that was originally identified in an in vivo screening of phage display libraries in tumor-bearing mice. iRGD is able to home to tumor tissues and has been used for its bifunctional action: homing to tumors and specific binding to neuropilin-1 (NRP-1) receptor with subsequent activation of a trans-tissue pathway for penetration into tumors. The RGD motif mediates binding to certain ay integrins expressed on tumor neovasculature and cancer cells. Upon binding, a protease cleavage event is activated, revealing a c-terminal motif (R/KXXR/K) in the peptide. This c-terminal motif then can bind to neuropilin-1 and activate an endocytotic/exocytotic transport pathway (formation of macropinosome-like vesicles that carry the peptide and bystander drugs into the deeper layers of tumor cells) that can be used to enhance transport of coupled and coadministered anti-cancer drugs into tumors. Thus, iRGD enhances tumor-specific cytotoxicity of almost any kind of co-injected cancer chemotherapeutic drug. See Sugahara et al., 2009, 2010; Pang et al., 2014; and U.S. Pat. No. 9,115,170 for further discussion on these topics.

However, the work presented in this application has shown that iRGD peptides have unexpected immunomodulating effects on their own, namely that they are able, unexpectedly, to potentiate the effects of immune checkpoint inhibitors in a manner that would not have been expected based on their previously known effects. iRGD peptides unexpectedly are able to deplete or suppress Tregs in a tumor-specific manner, which potentiates the effects of immune checkpoint inhibitors.

Disclosed are methods and compositions related to an isolated peptide comprising an amino acid segment comprising the amino acid sequence of SEQ ID NO: 2, or a variant thereof. In a specific example a variant relates a peptide defined by SEQ ID NO:3.

In alternative embodiments, the iRGD peptide or variant can comprise a chimera of the amino acid sequence SEQ ID NO: 2 or SEQ ID NO:3. Such a chimera can be additive, where sequence of one sequence is added to another sequence, substitutional, where sequence of one sequence is substituted for sequence of another sequence, or a combination. The disclosed peptides can consist of the amino acid segment.

The iRGD peptide or variant can be, for example, non-circular, linear, circular or cyclic. The amino acid segment can be circularized or cyclized via any suitable linkage, for example, a disulfide bond. The peptide can have any suitable length, such as a length of less than 100 residues. The peptide can have a length of, for example, less than 50 residues. The peptide can have a length of, for example, less than 20 residues.

Also disclosed are iRGD peptidomimetics that may be used in accord with the methods and compositions embodiments taught herein.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent than those discussed above. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Also disclosed are chimeric proteins containing a disclosed peptide fused to a heterologous protein. In one embodiment, the heterologous protein can have a therapeutic activity such as immune checkpoint inhibition activity, cytokine activity, cytotoxic activity or pro-apoptotic activity. In a further embodiment, the heterologous protein can be an antibody or antigen-binding fragment thereof. In other embodiments, the chimeric protein includes a peptide containing the amino acid sequence SEQ ID NO: SEQ ID NO: 2 or SEQ ID NO: 3, or a peptidomimetic thereof, fused to a heterologous protein. The term “heterologous,” as used herein in reference to a protein fused to the disclosed peptides, means a protein derived from a source other than the gene encoding the peptide or from which the peptidomimetic is derived. The disclosed chimeric proteins can have a variety of lengths including, but not limited to, a length of less than 100 residues, less than 200 residues, less than 300 residues, less than 400 residues, less than 500 residues, less than 800 residues or less than 1000 residues.

As used herein, “chimera” and “chimeric” refer to any combination of sequences derived from two or more sources. This includes, for example, from single moiety of subunit (e.g., nucleotide, amino acid) up to entire source sequences added, inserted and/or substituted into other sequences. Chimeras can be, for example, additive, where one or more portions of one sequence are added to one or more portions of one or more other sequences; substitutional, where one or more portions of one sequence are substituted for one or more portions of one or more other sequences; or a combination. “Conservative substitutional chimeras” can be used to refer to substitutional chimeras where the source sequences for the chimera have some structural and/or functional relationship and where portions of sequences having similar or analogous structure and/or function are substituted for each other. Typical chimeric and humanized antibodies are examples of conservative substitutional chimeras.

Also disclosed are bifunctional peptides which contains a iRGD peptide fused to a second peptide having a separate function. Such bifunctional peptides have at least two functions conferred by different portions of the full-length molecule and can, for example, display cytotoxic activity and immunomodulatory activity.

In one example, the iRGD peptide, chimera or bifunctional peptide can be circularized or cyclized via a disulfide bond. As used herein in reference to a peptide, the term “cyclic” means a structure including an intramolecular bond between two non-adjacent amino acids or amino acid analogues. The cyclization can be effected through a covalent or non-covalent bond. Intramolecular bonds include, but are not limited to, backbone to backbone, side-chain to backbone and side-chain to side-chain bonds. A preferred method of cyclization is through formation of a disulfide bond between the side-chains of non-adjacent amino acids or amino acid analogs. Residues capable of forming a disulfide bond include, for example, cysteine (Cys), penicillamine (Pen), β,β-pentamethylene cysteine (Pmc), β,β-pentamethylene-β-mercaptopropionic acid (Pmp) and functional equivalents thereof.

A peptide also can cyclize, for example, via a lactam bond, which can utilize a side-chain group of one amino acid or analog thereof to form a covalent attachment to the N-terminal amine of the amino-terminal residue. Residues capable of forming a lactam bond include aspartic acid (Asp), glutamic acid (Glu), lysine (Lys), ornithine (orn), α,β-diamino-propionic acid, γ-amino-adipic acid (Adp) and M-(aminomethyl)benzoic acid (Mamb).

Cyclization additionally can be effected, for example, through the formation of a lysinonorleucine bond between lysine (Lys) and leucine (Leu) residues or a dityrosine bond between two tyrosine (Tyr) residues. The skilled person understands that these and other bonds can be included in a cyclic peptide.

### (E.2) Conjugates

Disclosed are conjugates comprising a moiety and an iRGD peptide or peptide variant as defined herein. The moiety conjugated to an iRGD peptide or peptide variant can be any molecule. For example, moieties that affect the target, such as moieties with therapeutic effect, or that facilitate detection, visualization or imaging of the target, such as fluorescent molecule or radionuclides are conjugated to an iRGD peptide or peptide variant. In a specific example, disclosed are conjugates containing a chemotherapeutic agent linked to a iRGD peptide. It is believed that though the data presented herein demonstrates effects without the need for conjugating iRGD, an iRGD conjugate will provide synergistic effects with immune checkpoint inhibitors.

iRGD peptides can be usefully combined with, for example, moieties that can, for example, promote treat cancer, wound healing, anti-inflammatories, or analgesics. A variety of therapeutic agents are useful in the conjugates including, without limitation, a moiety that is an adjunct chemotherapeutic agent, anti-angiogenic agent, a pro-angiogenic agent, a cytotoxic agent, an anti-inflammatory agent, an anti-arthritic agent, a polypeptide, a nucleic acid molecule, a small molecule, a fluorophore, fluorescein, rhodamine, a radionuclide, indium-111, technetium-99, carbon-11, carbon-13, or a combination.

A conjugate containing multiple iRGD peptide molecules can include, for example, two or more, three or more, five or more, ten or more, twenty or more, thirty or more, forty or more, fifty or more, 100 or more, 200 or more, 300 or more, 400 or more, 500 or more, or 1000 or more iRGD peptide molecules. Moieties useful in a conjugate incorporating multiple iRGD peptide molecules include, without limitation, phage, retroviruses, adenoviruses, adeno-associated viruses and other viruses, cells, liposomes, polymeric matrices, non-polymeric matrices, particles (e.g. microparticles or nanoparticles) such as gold particles, microdevices, nanodevices, and nano-scale semiconductor materials.

A conjugate can contain, for example, a liposome or other polymeric matrix linked to at least two iRGD peptide molecules. If desired, the liposome or other polymeric matrix can be linked to at least ten, at least 100 or at least 1000 iRGD peptide molecules. Liposomes can be useful in such conjugates; liposomes consist of phospholipids or other lipids, are nontoxic, physiologically acceptable and metabolizable carriers that are relatively simple to make and administer (Gregoriadis, Liposome Technology, Vol. 1 (CRC Press, Boca Raton, Fla. (1984)). The liposome or other polymeric matrix can optionally include another component such as, without limitation, a therapeutic agent, adjunct cancer therapeutic agent, cytotoxic agent, anti-angiogenic agent, polypeptide or nucleic acid molecule.

Components of the disclosed conjugates can be combined, linked and/or coupled in any suitable manner. For example, moieties and iRGD peptide molecules can be associated covalently or non-covalently, directly or indirectly, with or without a linker moiety.

(E.3). Moieties

Disclosed are compositions and methods of directing a moiety to a target. As used herein, the term “moiety” is used broadly to mean a physical, chemical, or biological material that generally imparts a biologically useful function to a linked molecule. A moiety can be any natural or nonnatural material including, without limitation, a biological material, such as a cell, phage or other virus; an organic chemical such as a small molecule; a radionuclide; a nucleic acid molecule or oligonucleotide; a polypeptide; or a peptide. Useful moieties include, yet are not limited to an anti-angiogenic agent, a pro-angiogenic agent, an adjunct cancer therapeutic agent, an antibody, a cytotoxic agent, an anti-inflammatory agent, an anti-arthritic agent, a polypeptide, a nucleic acid molecule, a small molecule, a fluorophore, fluorescein, rhodamine, a radionuclide, indium-111, technetium-99, carbon-11, carbon-13, or a combination. Useful moieties further include, without limitation, phage and other viruses, cells, liposomes, polymeric matrices, non-polymeric matrices or particles such as gold particles, microdevices and nanodevices, and nano-scale semiconductor materials. These and other moieties known in the art can be components of a conjugate.

F. Combination Immunomodulatory Treatments for Cancer

Combination treatment with an immune checkpoint inhibitor and an iRGD peptide and/or iRGD conjugate is useful for treating any cancer that expresses inhibitory immune checkpoint surface molecules as discussed above. The treatment involves administration of the two types of agents in a coordinated manner so as to enhance the efficacy of an immune checkpoint inhibitor or reduce the likelihood of resistance or nonresponsiveness to treatment with an immune checkpoint inhibitor that is also administered to the subject. Administration can be simultaneous or sequential.

These inventive methods result in an approach to sensitize cancers such as PDAC to immunotherapy by unexpectedly potentiating the effects of immune checkpoint inhibitors that suppress the immune checkpoint molecules that turn off or decrease immune responses. Resistance to immunotherapy is a major issue in the treatment of various cancers. While various approaches have been tested, none of them have been proven effective to date, especially for PDAC. These inventive methods provide a solution to immunotherapy resistance and increase response to immunotherapy.

iRGD has shown no toxicity. It has been discovered that the iRGD tumor-penetrating peptide has immune modulating effects that can potentiate immune checkpoint inhibitors and thereby allow immune checkpoint inhibitors to be effective in the case of resistance and to have improved efficacy. The inventive methods allow iRGD to act as an adjuvant for a standard-of-care chemotherapy for various malignancies, such as PDAC, melanoma, ovarian cancer, brain, breast, lung, liver, bile duct, GI tract, prostate, uterine cancers, mesothelioma, sarcoma, and the like. The tumors can ne primary, metastatatic, or locally recurrent tumors.

The methods of the invention take advantage of iRGD's cancer-specific immunomodulatory effect to enhance sensitization of the cancer to immune checkpoint therapy agents by administering an iRGD peptide or variant thereof along with an immune checkpoint inhibitors. This technology depletes Tregs selectively in the cancer tissue, enhancing the efficacy of immunotherapy only against the cancer, without affecting the immune system in the entire body by generalized effects on Tregs. In summary, the combination treatment (addition of iRGD to immune checkpoint inhibitor immunotherapy) of the invention can avoid non-specific depletion of Tregs, which can lead to a series of inflammatory side effects. This combination therapy can sensitize a wide variety of cancers and can be administered at the same time as or sequential to traditional cancer treatment such as systemic chemotherapy, radiation, surgery, or other immunotherapy, since the inventive combination therapy does not kill tumor cells directly, but allows the natural immune defenses to work against the cancer to maximum benefit.

In some embodiments of the invention, combination treatment with an immune checkpoint inhibitor and an iRGD peptide or variant thereof comprises pre-treatment with the iRGD peptide prior to treatment with an immune checkpoint inhibitor, or vice versa. The treatments preferably overlap, such that both components of the combination treatment are present in the body of the subject at the same time.

When two or more agents (e.g., compounds or compositions) are used or administered “in combination” with each other, also referred to as “combination therapy” or “co-administration” they may be given at the same time, within overlapping time periods, or sequentially (e.g., separated by up to 2-4 weeks, 4-6 weeks, 6-8 weeks, or 8-12 weeks, in time), at least once, in various embodiments. The agents may be administered in the same composition or can be administered separately but sufficiently closely in time so as to provide the desired therapeutic effect. Preferably, the two components of the combination are present in the body of the subject to be treated at the same time or at least at overlapping times (i.e. overlapping biological effects of the administered agents). A person of ordinary skill in the art would readily determine appropriate timing, sequence, and dosages of administration for particular agents and compositions described herein.

Either or both of the components of the combination can be applied repeatedly, and different time intervals may be used over a course of treatment. There may be one or more cycles of administration of a first agent, followed by one or more cycles of administration of a second agent, and such cycles can be repeated one or more times. Agents administered in combination may be administered via the same route or different routes in various embodiments. They may be administered in either order in various embodiments. In some embodiments an agent is administered at least once between two doses of another agent. In some embodiments an agent is administered at least once between every second, third, or fourth dose of another agent. In some embodiments, agents are administered within 4, 8, 12, 24, 48, 72, or 96 hours of each other at least once. In some embodiments, agents are administered within 4, 8, 12, 24, 48, 72, or 96 hours of each other multiple times. In some embodiments, a first agent is administered prior to or after administration of the second agent, e.g., sufficiently close in time that the two agents are present together at useful levels within the body at least once. In some embodiments, the agents are administered sufficiently close together in time such that no more than 50%, 75%, or 90% of the earlier administered agent has been metabolized to inactive metabolites or eliminated, e.g., excreted, from the body, at the time the second agent is administered.

Combination therapy with an immune checkpoint inhibitor and an iRGD peptide or variant thereof results in increased immune-mediated destruction of tumors and improve the rate of overall tumor response and duration of response compared to administration of the immune checkpoint inhibitor alone. This type of effect can contribute to an improvement in overall survival for the patient compared to treatment using an immune checkpoint inhibitor alone.

Overall survival can be measured as the median survival following the initiation of treatment with the immune checkpoint inhibitor. Overall survival can additionally or alternately be measured as the overall survival rate at, e.g., 1 month, 2 months, 3 months, 4 months, 6 months, 9 months, 12 months (1 year), 18 months, 2 years, 3 years, 4 years, 5 years, etc., following the initiation of treatment with the immune checkpoint inhibitor. In some embodiments, the overall survival rate at one or more of the afore-mentioned time points can be increased by at least 10%, 20%, 30%, 40%, 50%, 60%, 70%, 80%, 90%, 100%, 2.5-fold, 3-fold, 4-fold, 5-fold, or more, in subjects treated with an immune checkpoint inhibitor and an iRGD peptide as compared with subjects treated with the same immune checkpoint inhibitor but not treated with the iRGD peptide. In some embodiments, the overall survival rate at one or more of the afore-mentioned time points is increased by at least 10%, 20%, 30%, 40%, 50%, 60%, 70%, 80%, 90%, 100%, 2.5-fold, 3-fold, 4-fold, 5-fold, or more in subjects who are treated with an immune checkpoint inhibitor, an iRGD peptide, and one or more additional anti-cancer therapies, compared with subjects treated with the same immune checkpoint inhibitor and same additional anti-cancer therapies but not treated with the iRGD peptide. In some embodiments, the overall median survival may be increased by at least 1 month, 2 months, 3 months, 4 months, 6 months, 9 months, 12 months, 18 months, 2 years, or more, in subjects treated with an immune checkpoint inhibitor and an iRGD peptide as compared with subjects treated with the same immune checkpoint inhibitor but not treated with the iRGD peptide. In some embodiments, the overall survival rate at one or more of the afore-mentioned time points may be increased by at least 1 month, 2 months, 3 months, 4 months, 6 months, 9 months, 12 months, 18 months, 2 years, or more in subjects who are treated with an immune checkpoint inhibitor, one or more additional anti-cancer therapies, and a an iRGD as compared with subjects treated with the same immune checkpoint inhibitor and same additional anti-cancer therapies but not treated with the iRGD peptide.

G. Suitable Cancers for Treatment

In terms of the present invention, preferred cancers for these treatments include immune checkpoint inhibitor sensitive and immune checkpoint inhibitor resistant cancers, including cancers that previously have responded to immune checkpoint inhibitor treatments but have become refractory. Methods described herein may, in general, be used with regard to any type of cancer.

A variety of different tumor types can arise in certain organs. These can differ with regard to clinical and/or pathological features and molecular markers. Tumors arising in a variety of different organs are described in the WHO Classification of Tumours series, 4th ed., or 3rd ed. (Pathology and Genetics of Tumours series), by the International Agency for Research on Cancer (IARC), WHO Press, Geneva, Switzerland, all volumes of which are incorporated herein by reference. Extensive information regarding different types of cancer and their diagnosis and treatment may be found in DeVita, V T, et al., DeVita, Hellman, and Rosenberg's Cancer: Principles and Practice of Oncology (Cancer: Principles & Practice, Lippincott, Williams, and Wilkins, 9th ed. (2011).

In certain embodiments of the invention, the cancer type is one for treatment of which an immune checkpoint inhibitor has been tested in at least one Phase I trial and resulted in responses in at least some subjects. In certain embodiments the cancer type is one for treatment of which an immune checkpoint inhibitor has been tested in at least one Phase II trial and resulted in responses in at least some subjects. In certain embodiments the cancer type is one for treatment of which an immune checkpoint inhibitor has been tested in at least one Phase III trial and resulted in responses in at least some subjects. In certain embodiments the cancer type is one for treatment of which an immune checkpoint inhibitor has been approved for use by the US Food & Drug Administration (FDA), the European Medicines Agency (EMA), or both.

A subject who is to be treated or is being treated for a cancer may be one whom a medical practitioner has diagnosed as having such a condition. In some embodiments the subject may be or may have been monitored for the cancer and/or for response to treatment. Diagnosis and/or monitoring may be performed by any appropriate means and may involve, for example, detecting a mass on physical examination, by imaging (e.g., X-ray, CT scan, MRI scan, PET scan, ultrasound), histopathological examination of a biological sample or other means of detecting cancer cells or cancer cell products (e.g., tumor antigens), detecting symptoms associated with cancer. In some embodiments the subject may have exhibited progressive disease or recurrence despite treatment with one or more conventional anti-cancer agents, radiotherapy, or combination thereof. In some embodiments the patient may have exhibited progressive disease or recurrence despite treatment with one or more molecularly targeted anti-cancer agents and/or radiotherapy.

In certain embodiments of the invention, the cancer is metastatic, unresectable, or both. In some embodiments the cancer is a Stage III, IIIb, or Stage IV cancer. Cancer stages may be assigned based on the TNM system, described in Sobin L H, Gospodarowicz M K, Wittekind Ch. Eds. TNM Classification of Malignant Tumors, 7th ed. Wiley-Blackwell, Oxford 2009 or in the American Joint Commission on Cancer (AJCC Cancer Staging Manual, Eds. Edge et al., Springer, 7th edition, 2010. Stage I and Stage II cancers also may be treated.

Therefore, the invention is suitable for use with any cancer, including but not limited to breast cancer, biliary tract cancer, bladder cancer, brain cancer (e.g., glioblastomas, medulloblastomas), cervical cancer, choriocarcinoma, colon cancer, endometrial cancer, esophageal cancer, gastric cancer, head and neck cancer, hematological neoplasms (including acute lymphocytic leukemia and acute myelogenous leukemia, T-cell acute lymphoblastic leukemia/lymphoma, hairy cell leukemia, chronic lymphocytic leukemia, chronic myelogenous leukemia, multiple myeloma, adult T-cell leukemia/lymphoma), intraepithelial neoplasms (including Bowen's disease and Paget's disease), liver cancer, lung cancer (including non-small cell lung cancer and small cell lung cancer), lymphomas (including Hodgkin's disease and lymphocytic lymphomas), malignant mesothelioma, melanoma (including metastatic melanoma), neuroblastoma, oral cancer (including squamous cell carcinoma), ovarian cancer (including ovarian cancer arising from epithelial cells, stromal cells, germ cells and/or mesenchymal cells), pancreatic cancer, prostate cancer, rectal cancer, renal cell cancer, sarcomas (including angiosarcoma, gastrointestinal stromal tumors, leiomyosarcoma, rhabdomyosarcoma, liposarcoma, fibrosarcoma, and osteosarcoma), renal cancer (including renal cell carcinoma and Wilms tumor), skin cancer (including basal cell carcinoma and squamous cell cancer), stomach cancer, testicular cancer (including germinal tumors such as seminoma, non-seminoma (teratomas, choriocarcinomas), stromal tumors, and germ cell tumors), and thyroid cancer (including thyroid adenocarcinoma and medullary carcinoma).

Immune checkpoint inhibitors are known to treat a variety of cancer types, including but not limited to pancreatic duct adenocarcinoma, breast cancer, bladder cancer, cervical cancer, colon cancer, head and neck cancer, Hodgkin lymphoma, liver cancer, lung cancer, renal cell cancer, skin cancer, stomach cancer, rectal cancer, and any solid tumor that is not able to repair errors in its DNA that occur when the DNA is copied. Therefore, any of these cancers are contemplated for use with this invention and are preferred. Additionally, preferred cancers for treatment with embodiments according to the invention harbor αv integrin+ NRP-1+ or 2, Tregs such as ovarian cancer, melanoma, or pancreatic duct adenocarcinoma. Any subject suffering from any of these cancers, or the cancers enumerated in the preceding paragraph is contemplated to benefit from the methods of the invention described here. A highly preferred cancer for the inventive treatment methods is pancreatic duct adenocarcinoma.

H. Additional Treatment Components

In some embodiments of the invention, an additional anti-cancer treatment modality is administered in combination (simultaneously or sequentially) with both an immune checkpoint inhibitor and an iRGD peptide. Any of a wide variety of anti-cancer agents can be used as are known in the art. The particular additional agent may be selected based on, e.g., the particular tumor to be treated by the person of skill.

Anti-cancer agents suitable for use in the invention include, but are not limited to, surgery, radiotherapy, chemotherapy (drug therapy), or immunotherapy. Anti-cancer agents include a variety of different types of agents, including antibodies, polypeptides, and small molecules. Non-limiting examples of cancer chemotherapeutic agents that may be used include, e.g., alkylating and alkylating-like agents such as nitrogen mustards (e.g., bendamustine, chlorambucil, chlormethine, cyclophosphamide, ifosfamide, uramustine, and melphalan), busulfan, dacarbazine, procarbazine, temozolomide, thioTEPA, treosulfan, nitrosoureas (e.g., carmustine, fotemustine, lomustine, streptozocin); platinum agents (e.g., alkylating-like agents such as carboplatin, cisplatin, oxaliplatin, satraplatin, trinuclear platinum compounds such as BBR3464 and DH6Cl); antimetabolites such as folic acids (e.g., aminopterin, methotrexate, pemetrexed, raltitrexed); purines such as cladribine, clofarabine, fludarabine, mercaptopurine, pentostatin, thioguanine; pyrimidines such as capecitabine, cytarabine, fluorouracil, floxuridine, gemcitabine; spindle poisons/mitotic inhibitors such as taxanes (e.g., docetaxel, paclitaxel), vincas (e.g., vinblastine, vincristine, vindesine, and vinorelbine), epothilones; cytotoxic/anti-tumor antibiotics such anthracyclines (e.g., daunorubicin, doxorubicin, epirubicin, idarubicin, mitoxantrone, pixantrone, and valrubicin), compounds naturally produced by various species of Streptomyces (e.g., actinomycin, bleomycin, mitomycin, plicamycin) and hydroxyurea; topoisomerase inhibitors such as camptotheca (e.g., camptothecin, topotecan, irinotecan) and podophyllums (e.g., etoposide, teniposide); monoclonal antibodies for cancer therapy such as anti-receptor tyrosine kinases (e.g., cetuximab, panitumumab, trastuzumab), anti-CD20 (e.g., rituximab, ofatumumab, and tositumomab), anti-CD19 (e.g., blinatumomab) and others for example alemtuzumab (an anti-CD52 antibody), gemtuzumab; photosensitizers such as aminolevulinic acid, methyl aminolevulinate, porfimer sodium, and verteporfin; tyrosine and/or serine/threonine kinase inhibitors, e.g., inhibitors of Abl, Kit, insulin receptor family member(s), VEGF receptor family member(s), EGF receptor family member(s), PDGF receptor family member(s), FGF receptor family member(s), mTOR, Raf kinase family, phosphatidyl inositol (PI) kinases such as PI3 kinase, PI kinase-like kinase family members, MEK, cyclin dependent kinase (CDK) family members, Aurora kinase family members (e.g., kinase inhibitors that are on the market or have shown efficacy in at least one phase III trial in tumors, such as cediranib, crizotinib, dasatinib, dabrafenib, erlotinib, gefitinib, imatinib, lapatinib, nilotinib, sorafenib, sunitinib, trametinib, vandetanib, vemurafenib), growth factor receptor antagonists; retinoids (e.g., alitretinoin and tretinoin); altretamine; amsacrine; anagrelide; arsenic trioxide; asparaginase (e.g., pegasparagase); bexarotene; proteasome inhibitors such as bortezomib or carfilzomib; denileukin diftitox; estramustine; ixabepilone; masoprocol; mitotane; testolactone; Hsp90 inhibitors; angiogenesis inhibitors, e.g., anti-vascular endothelial growth factor agents such as bevacizumab (Avastin) or VEGF receptor antagonists or soluble VEGF receptor domain (e.g., VEGF-Trap); matrix metalloproteinase inhibitors, etc. See also Table 1, below, for selected adjunct cancer therapeutic agents.

TABLE 1
Selected Adjunct Cancer Therapeutic Agents.

| | | |
|---|---|---|
| Abiraterone acetate | Becenum | Cevarix |
| Abitrexate | Beleodaq | Cetuximab |
| Abraxane | Belinostat | Chlorambucil |
| ABVD | Bendamusine hydrochloride | Chlorambucil-prednisone |
| ABVE | BEP | CHOP |
| ABVE-PC | Bevacizumab | Cisplatin |
| AC | Bexarotene | Clafen |
| AC-T | Bexxar | Clofarabine |
| Adcetris | Bicalutamide | Clofarex |
| ADE | BiCNU | Clolar |
| Ado-Trastuzumab Emtansine | Bleomycin | CMF |
| Adriamycin | Blinatumomab | Cometriz |
| Adrucil | Blincyto | COPP |
| Afatinib | Bortezomib | COPP-ABV |
| Afinitor | Bosulif | Cosmegen |
| Aldara | Bosutnib | Crizotinib |
| Aslesleukin | Brentuximab Vedotin | CVP |
| Alemtuzumab | Busulfan | Cyclophosphamide |
| Alimta | Busulfex | Cyfos |
| Aloxi | Cabazitaxel | Cyramza |
| Ambochlorin | Cabozantinib-S-malate | Cytarabine |
| Aminolevulinic acid | CAF | Cytarabine, liposomal |
| Anastrozole | Campath | Cytosar-U |
| Aprepitant | Camptosar | Cytoxan |
| Aredia | Capecitabine | Dabrafenib |
| Arimidex | CAPOX | Dacarbazine |
| Aromasin | Carboplatin | Dacogen |
| Arranon | Carboplatin-Taxol | Dactinomycin |
| Arsenic trioxide | Carfilzimib | Dasatinib |
| Arserra | Carmubris | Daunorubicin hydrochloride |
| Asparaginase erwinia | Carmustine | Decitabine |
| chrysanthemi | Carmustine implant | Degarelix |
| Avastin | Casodex | Denileudin diftitox |
| Axitinib | CeeNU | Denosumab |
| Azacitidine | Ceritinib | Dexrazoxane hydrochloride |
| BEACOPP | Ceruvibidine | Dinutuximab |
| DepoCyt | Gilotrif | Lupron depot |
| Depofoam | Gleevec | Lupron depot-Ped |
| Dexrazoxane hydrochloride | Gliadel | Lupron depot-3 month |
| Docetaxel | Gliadel wafer | Lupron depot-4 month |
| Doxil | Glucarpidase | Lynparza |
| Doxorubicin hydrochloride | Goserelin acetate | Marqibo |
| Doxorubicin hydrochloride | Halaven | Matulane |
| liposome | Herceptin | Mechlorethamine |
| Dox-SL | HPV bivalent vaccine, recomb | hydrochloride |
| DTIC-Dome | HPV nonavalent vaccine, | Megace |
| Efudex | recomb | Megestrol acetate |
| Elitek | HPV quadrivalent vaccine, | Mekinist |
| Ellence | recomb | Mercaptopurine |
| Eloxatin | Hycamtin | Mesna |
| Eltrombopag olamine | Hyper-CVAD | Mesnx |
| Emend | Ibrance | Methazolastone |
| Enzalutamide | Ibritumomab Tiuxetan | Methotrexate |
| Epirubidin hydrochloride | Ibrutinib | Methotrexate LPF |
| EPOCH | ICE | Mexate |
| Erubitux | Iclusig | Mexate-AQ |
| Eribulin mesylate | Idamycin | Mitomycin C |
| Erivedge | Idamycin hydrochloride | Mitoxantrone hydrochloride |
| Erloinib | Idelalisib | Mitozytrex |
| Erwinaze | Ifex | MOPP |
| Etoposide | Ifosfamide | Mozobil |
| Etoposide phosphate | Ifosfamidum | Mustargen |
| Evacet | Imatinib | Mutamycin |
| Everolimus | Imbruvica | Myleran |
| Evista | Imiquimod | Mylosar |
| Exemestane | Inlyta | Mylotarg |
| Fareston | Intron A | Nanoparticle paclitaxel |
| Farydak | Iodine 131 Tositumomab | Navelbine |
| Faslodex | Ipilimumab | Nelarabine |
| FEC | Iressa | Neosar |
| Femara | Irinotecan hydrochloride | Neupogen |
| Filgrastim | Istodax | Nexavar |
| Fludara | Ixabepilone | Nilotinib |
| Fludarabine phosphate | Ixempra | Nivolumab |
| Fluoroplex | Jakafi | Nolvadex |
| Fluorouracil | Jevtana | Nplate |
| Folex | Kadcyla | Obinutuzumab |
| Folex PFS | Keoxifene | OEPA |
| FOLFIRI | Kepivance | Ofatumumab |
| FOLFIRI-BEVACIZUMAB | Keytruda | OFF |
| FOLFIRI-CETUXIMAB | Kyprolis | Olaparib |
| FOLFIRINOX | Lanreotide acetate | Omacetaxine mepesuccinate |
| FOLFOX | Lpaptinib ditosylate | Oncaspar |
| Folotyn | Lenalidomide | Ontak |
| FU-LV | Lenvatinib mesylate | Opdivo |
| Fulvestrant | Lenvima | OPPA |
| Gardasil | Letrozole | Oxaliplatin |
| Gardasil 9 | Leucovorin calcium | Paclitaxel |
| Gazyva | Leukeran | Paclitacel albumin stabilized |
| Gefitinib | Leuprolide | nanoparticle formulation |
| Gemcitabine hydrochloride | Lefulan | PAD |
| GEMCITABINE-CISPLATIN | Linfolizin | Palbociclib |
| GEMCITABINE- | LipoDox | Palifermin |
| OXALIPLATIN | Liposomal Cytarabine | Palonosetron hydrochloride |
| Gentuzumab Ozogamicin | Lomustine | Pamidronate disodium |
| Gemzar | Lupron | Panitumumab |
| Panobinostat | Ruxolitinib phosphate | Tykerb |
| Paraplat | Sclerosol intrapleural aerosol | Unituxin |
| Paraplatin | (talc) | VAMP |
| Pazipanib hydrochloride | Siltuximab | Vandetanib |
| Pegaspargase | Sipuleucel-T | Vectibix |
| Peginterferon Alfa-2b | Somatuline depot | VelP |
| PEG-Intron | Sorafenib tosylate | Velban |
| Pembrolizumab | Sprycel | Velcade |
| Pemetrexed disodium | STANFORD V | Velsar |
| Perjeta | Sterile Talc Powder | Vemurafenib |
| Pertuzumab | Steritalc | VePesid |
| Platinol | Stivarga | Viadur |
| Platinol-AQ | Sunitinib malate | Vidaza |
| Plerixafor | Sutent | Vinblastine sulfate |
| Pomalidomide | Sylatron | Vincasar PFS |
| Pomalyst | Sylvan | Vincristine sulfate |
| Ponatinib | Synovir | Vincristine sulfate liposome |
| Pralatexate | TAC | Vinorelbine tartrate |
| Prednisone | Tafinlar | VIP |
| Procarbazine hydrochloride | Talc | Vismodegib |
| Proleukin | Tamoxifen citrate | Voraxaze |
| Prolia | Tarabine PFS | Vorinostat |
| Promacta | Tarceva | Votrient |
| Provenge | Targretin | Wellcovorin |
| Purinethol | Tasigna | Xalkori |
| Radiuim 223 dichloride | Taxol | XELIRI |
| Raloxifene hydrochloride | Taxotere | Xeloda |
| Ramucirumab | Temodar | XELOX |
| Rasburicase | Temozolomide | Xgeva |
| R-CHOP | Temsirolimus | Xofigo |
| R-CVP | Thalidomide | Xtandi |
| R HPV bivalent vaccine | Thalomid | Yervoy |
| R HPV nonavalent vaccine | Thiotepa | Zaltrap |
| R HPV quadrivalent vaccine | Toposar | Zelboraf |
| Recomb Interferon Alfa-2b | Topotecan hydrochloride | Zevalin |
| Regorafenib | Toremifene | Zinecard |
| R-EPOCH | Torisel | Ziv-aflibercept |
| Revlimid | Tositomomab | Zoladex |
| Rheumatrex | Totect | Zoledronic acid |
| Rituxan | TPF | Zolinza |
| Rituximab | Trametinib | Zometa |
| Romidepsin | Trastusumab | Zydelig |
| Romiplostim | Treanda | Zykadia |
| Rubidomycin | Trisenox | Zytiga |

I. Other Treatments

A variety of infections also are characterized by a state of immune dysfunction, e.g., anergy or exhaustion, mediated at least in part by immune checkpoint pathways Immune checkpoint inhibitors also can be useful in treating such disorders. For example, signaling through PD-1 attenuates T cell antigen receptor signals and inhibits the cytokine production and cytolytic function of T cells, both in cancer and in chronic infections. Blockade of PD-1 or PD-L1 during chronic viral infection can restore T cell function and diminish the viral load. iRGD peptides can potentiate this effect as well. Some embodiments of the invention therefore include a method of treating a subject in need of an enhanced immune response due to cancer, chronic infection, or chronic inflammatory disease comprising treating the subject in combination with an immune checkpoint inhibitor and an iRGD peptide.

In some embodiments, the subject is one in whom an inhibitory immune checkpoint pathway is overactive as compared with a normal, healthy subject, for example a subject suffering from one or more of chronic infection, cancer, chronic inflammation. In some embodiments of the invention, methods include method of reducing or reversing immune cell dysfunction due to cancer or chronic infection in a subject in need thereof comprising treating the subject with an immune checkpoint inhibitor and an iRGD peptide. In some embodiments, the subject is a cancer patient who also suffers from a chronic infection or chronic inflammation. In some embodiments the subject does not have cancer but suffers from a chronic infection or chronic inflammation. A chronic infection is an infection that does not respond to conventional treatments with antibiotics or antivirals, or that keeps returning despite treatment. Such infections can occur in virtually any system, organ, or tissue in the body, and can include bacterial and viral infections.

J. Pharmaceutical Compositions, Dosage Forms, and Routes of Administration

In preferred method embodiments, the compounds described herein are formulated and are administered as one or more pharmaceutical compositions that include a pharmaceutically acceptable carrier and one or more pharmaceutical agent, including one or more of the inventive compounds described herein, and optionally including one or more of the inventive compounds described herein with an additional agent. A pharmaceutically acceptable carrier refers to any convenient compound or group of compounds that is not toxic to the subject and that does not destroy or significantly diminish the pharmacological activity of the therapeutic agent(s) with which it is formulated. Such pharmaceutically acceptable carriers or vehicles encompass any of the standard pharmaceutically accepted solid, liquid, or gaseous carriers known in the art, such as those discussed in the art.

A suitable carrier depends on the route of administration contemplated for the pharmaceutical composition. Routes of administration are determined by the person of skill according to convenience, the health and condition of the subject to be treated, and the location and stage of the condition to be treated, however the preferred route of administration for the methods of this invention is intravenous, either by injection or infusion. Other preferred routes of administration include direct injection into a particular area in need of treatment, such as an area of infection, a tumor or the area around a tumor, injection into a specific blood vessel that supplies or is located at least in part within an organ, tissue, or tumor to be treated, mucosal administration with a mucoadhesive carrier system, intraarterial injection, intrathecal injection, subcutaneous injection, intramuscular injection, and the like. Immune checkpoint inhibitors preferably are administered intravenously by injection or infusion, including bolus injection, or intermittent or continuous infusion, e.g., using an infusion pump, etc.

iRGD peptides preferably also are administered intravenously by injection or infusion.

For intravenous administration, a liquid or semi-liquid carrier is most often used, including a solution or suspension. The forms which the pharmaceutical composition can take can include, but are not limited to: liquids, powders or granules for dilution, solutions, suspensions, emulsions, dispersions, lipid vesicles, oils, gels, and the like, for example aqueous solutions (e.g., physiological saline solutions, phosphate-buffered saline solutions, Ringer's, sodium acetate or potassium acetate solution, 5% dextrose, and the like), oil-in-water or water-in-oil emulsions. The carrier also can contain one or more of ethanol, glycerol, propylene glycol, water, a carbohydrate (e.g., glucose, sucrose, lactose), dextrans, amino acids (e.g., glycine), polyols (e g, mannitol, a diluent, a filler, a bulking agent, a solvent, a tonicity modifying agent, a buffer, a pH-modifying agent, a surfactant (e.g., Tween-80™, Pluronic-F108/F68™, deoxycholic acid, phosphatidylcholine), a preservative, an antioxidant, an emulsifier, a chelating agent, an antimicrobial (such as an antibacterial, antifungal, or bacteriostatic compound), agent(s) to produce delayed absorption and/or any other additional compound or material, as desired. Persons of skill in the art are well aware of such compounds and can select any such excipients which are convenient and known in the art. One of ordinary skill in the art will be aware of numerous physiologically acceptable compounds that may be included in a pharmaceutical composition.

Preferably, the compositions for injection are sterile, acceptably free of endotoxin, and are sufficiently fluid for easy use in a syringe. In addition, the composition should be stable under the conditions of manufacture and storage. The pharmaceutical compositions optionally are contained in a package or kit. Packages can include multiple dose vials, ampoules, pre-filled syringes, infusion bags, boxes, bottles, and the like, and may include instructions for use.

In preferred embodiments, pharmaceutical compositions are sterile solutions prepared by incorporating one or more of the active compounds in the required amount in an appropriate solvent, optionally with one or a combination of ingredients such as buffers (e.g., acetates, citrates, lactates or phosphates), agents for the adjustment of tonicity (e.g., sodium chloride or dextrose), antibacterial agents (e.g., benzyl alcohol or methyl parabens), antioxidants (e.g., ascorbic acid, glutathione, or sodium bisulfate), chelating agents (e.g., EDTA), and other suitable ingredients etc., as desired, followed by filter-based sterilization.

Pharmaceutical compositions can be formulated to contain each of the components of the combination treatment alone, or can be formulated to contain both an immune checkpoint inhibitor and an iRGD peptide, optionally also including one or more additional treatment agents. Supplementary active compounds, e.g., compounds independently useful for treating a subject suffering from cancer or an infection, can also be incorporated into any of a pharmaceutical composition containing an immune checkpoint inhibitor, an iRGD peptide, or both. All of these pharmaceutical compositions also preferably other inert carriers or excipients as appropriate for the formulation desired, e.g., as discussed herein. Therefore, in certain embodiments the pharmaceutical compositions contain only one active agent each, and some pharmaceutical compositions are combinations of two or more active agents in one composition.

In some aspects, the invention described herein comprises a pharmaceutically acceptable immune checkpoint inhibitor or pharmaceutically acceptable composition comprising an immune checkpoint inhibitor, packaged together in a pharmaceutical pack or kit with a package insert (label) approved by a government agency responsible for regulating pharmaceutical agents, e.g., the FDA or EMA, wherein the label includes use of the immune checkpoint inhibitor in combination with an iRGD peptide.

K. Doses and Regimens

Treatment regimens suitable for the inventive methods include a single administration or a course of administrations lasting two or more days, including a week, two weeks, several weeks, a month, two months, several months, a year, or more, including administration for the remainder of the subject's life. The regimen can include multiple doses per day, one dose per day or per week, for example, or a long infusion administration lasting for an hour, multiple hours, a full day, or longer.

Dosage amounts per administration include any amount determined by the practitioner, and will depend on the size of the subject to be treated, the state of the health of the subject, the route of administration, the condition to be treated or prevented, and the like. In general, appropriate doses of immune checkpoint inhibitor, iRGD peptide, or other active agent also depend at least in part upon the potency of the agent and route of administration.

Dose ranges that are effective and well tolerated can be selected by one of ordinary skill in the art. Those of ordinary skill in the art will also understand that certain agents are typically used in combination with other therapies, and that an “effective amount” of such an agent for treating a disorder may be an amount such that the therapeutic effect of interest is produced by the combination of the agent and the other therapies, also used at their effective amounts. Optionally, a dose may be tailored to the particular recipient, for example, through administration of increasing doses until a preselected desired response is achieved. If desired, the specific dose level for any particular subject may be selected based at least in part upon a variety of factors including the activity of the specific compound employed, the particular condition being treated and/or its severity, the age, body weight, general health, route of administration, and/or any concurrent medication.

In some embodiments of the invention, an effective amount or dose of an immune checkpoint inhibitor ranges from about 0.001 to about 500 mg/kg body weight, e.g., about 0.01 to 100 mg/kg body weight, e.g., about 0.1 to about 50 mg/kg body weight, about 0.1 to about 20 mg/kg body weight, e.g., about 1 to about 10 mg/kg. In some embodiments of the invention, an effective amount may be between about 1 mg and about 10,000 mg, e.g., between about 1 mg and about 10 mg, between about 10 mg and about 100 mg, between about 100 mg and about 1000 mg, between about 1000 mg and about 2000 mg.

In some embodiments of the invention, the immune checkpoint inhibitor is administered about every 2-6 weeks, e.g., about every 2 weeks, about every 3 weeks, about every 4 weeks, or about every 6 weeks.

In some embodiments of the invention, an immune checkpoint inhibitor antibody is administered using an escalating dosage regimen including administering a first dosage at about 3 mg/kg, a second dosage at about 5 mg/kg, and a third dosage at about 9 mg/kg. Another escalating dosage regimen may include administering a first dosage of immune checkpoint inhibitor antibody about 3 mg/kg, a second dosage of about 3 mg/kg, a third dosage of about 5 mg/kg, a fourth dosage of about 5 mg/kg, and a fifth dosage of about 9 mg/kg. Specific exemplary dosages of immune checkpoint inhibitor antibodies include 3 mg/kg ipilimumab administered every three weeks for four doses; 10 mg/kg ipilimumab every three weeks for eight cycles; 10 mg/kg every three weeks for four cycles then every 12 weeks; 10 mg/kg MK-3475 every two or every three weeks; 2 mg/kg MK-3475 every three weeks; 15 mg/kg tremilimumab every three months; 0.1, 0.3, 1, 2, 3 or 10 mg/kg nivolumab every two weeks for up to 96 weeks (or longer); 0.3, 1, 3, or 10 mg/kg BMS-936559 every two weeks for up to 96 weeks (or longer) (Kyi C. & Postow, M A, FEBS Lett. (2014) 588(2):368-76; Callahan, M K & Wolchok, J D (2013) J Leukoc Biol 94:41-53); pembrolizumab at doses of 2 mg/kg and 10 mg/kg every 3 weeks (Hamid, O., N Engl J Med. 2013; 369(2):134-44); BMS-936559 at doses of 1 mg/kg, 3 mg/kg, or 10 mg/kg every 2 weeks (Brahmer, J R, et al., N Engl J Med 2012; 366:2455-65); pidilizumab at 3 mg/kg intravenously every 4 weeks (Westin, J R, et al., Lancet Oncol. 2014 January; 15(1):69-77).

In some embodiments a PD1 pathway inhibitor, e.g., a PD1 inhibitor, PD-L1 inhibitor, or PD-L2 inhibitor is used in an amount sufficient to decrease one or more biological activities of PD1 by at least 20%, 30%, 40%, 50%, 60%, 70%, 80%, 90%, 95%, or 100% relative to a suitable control. In some embodiments a PD1 pathway inhibitor decreases the biological activity of PD1 by reducing binding of PD1 to PD-L1, PD-L2, or both by at least 20%, 30%, 40%, 50%, 60%, 70%, 80%, 90%, 95%, or 100% relative to a suitable control. PD1 pathway inhibition, e.g., PD1 blockade, can be accomplished by a variety of mechanisms using any of a variety of agents, including, e.g., with antibodies or other agents that bind PD1 or its ligand(s), PD-L1 and/or PD-L2.

In some embodiments of the invention, a CTLA4 inhibitor is used in an amount sufficient to inhibit expression and/or decrease biological activity of CTLA4 by at least 20%, 30%, 40%, 50%, 60%, 70%, 80%, 90%, 95%, or 100% relative to a suitable control, e.g., between 50% and 75%, 75% and 90%, or 90% and 100%. In some embodiments a CTLA4 pathway inhibitor is used in an amount sufficient to decrease the biological activity of CTLA4 by reducing binding of CTLA4 to CD80, CD86, or both by at least 20%, 30%, 40%, 50%, 60%, 70%, 80%, 90%, 95%, or 100% relative to a suitable control, e.g., between 50% and 75%, 75% and 90%, or 90% and 100% relative to a suitable control. A suitable control in the context of assessing or quantifying the effect of an agent of interest is typically a comparable biological system (e.g., cells or a subject) that has not been exposed to or treated with the agent of interest, e.g., a CTLA4 pathway inhibitor (or has been exposed to or treated with a negligible amount). In some embodiments a biological system may serve as its own control (e.g., the biological system may be assessed before exposure to or treatment with the agent and compared with the state after exposure or treatment has started or finished. In some embodiments a historical control may be used.

In certain embodiments, an effective dose of a composition as described herein can be administered to a patient once. In certain embodiments, an effective dose of a composition as described herein can be administered to a patient repeatedly. For systemic administration, subjects can be administered a therapeutic amount of an iRGD containing composition as described herein, such as, e.g. 0.01 mg/kg, 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more of iRGD amount per weight of subject.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. tumor size and/or growth by at least 10%, at least 15%, at least 20%, at least 25%, at least 30%, at least 40%, at least 50%, at least 60%, at least 70%, at least 80% or at least 90% or more.

The dosage of iRGD or iRGD composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity a composition as described herein. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition as described herein can be administered over a period of time, such as over a 5 minute, 10 minute, 15 minute, 20 minute, or 25 minute period.

### 4. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: iRGD Therapy Increases CD8⁺ T Cells in Transgenic PDAC Mice

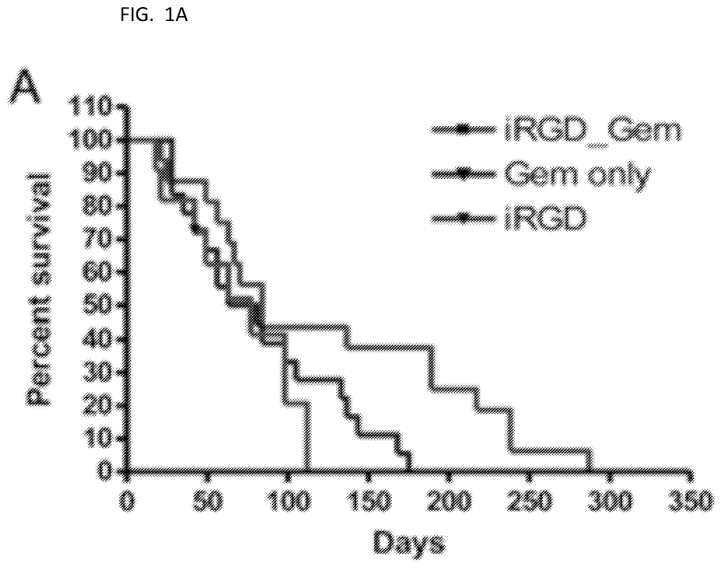

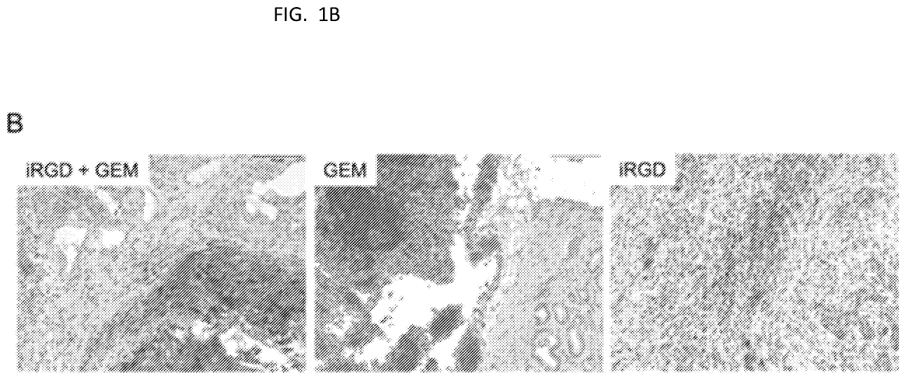

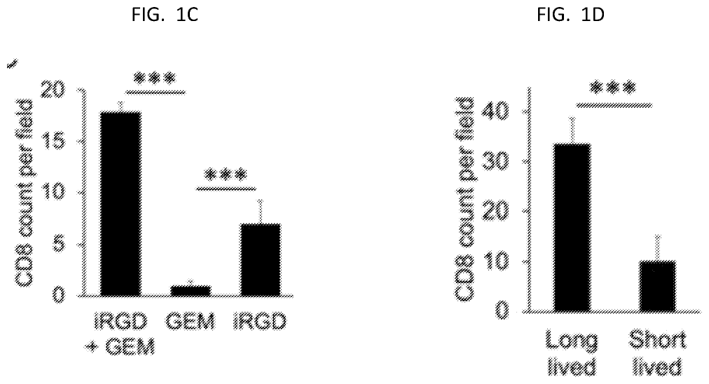

FIG. 1 presents data showing that iRGD therapy increases CD8⁺ T cells in transgenic PDAC mice. Long-term treatment with iRGD co-injection therapy significantly prolonged the survival of transgenic Kras_La^{GD12}, p53-LSL^{172H}, Pdx-1-cre (KPC) mice treated with GEM (see FIG. 1A). We randomized mice to therapy with intraperitoneal (IP) gemcitabine alone (100 mg/kg/injection), intravenous (IV) iRGD alone (100 μg/injection) or gemcitabine co-administered with iRGD. Mice were followed with high resolution ultrasound and manual palpation until at least one tumor nodule of 4-5 mm became both palpable and visible on consecutive ultrasound exams. At this point, the mice were randomized to one of the three treatment arms. Treatment was given every 4 days until the animals were sacrificed for having signs of distress. An interim analysis of the data revealed that iRGD treatment alone was unlikely to offer any therapeutic benefit based on literature and our previous observations, and that arm was discontinued. Ultimately, mice treated with the combination of iRGD and gemcitabine survived significantly longer than those treated with gemcitabine alone (hazard ratio=0.53, p=0.0385). Of note, while there was a difference in median survival favoring the combination (71 days versus 84 days), there was a more striking difference in the tail of the survival curve such that survival at 180 days was 0% for gemcitabine alone versus 40% in the iRGD+gemcitabine group.

Tumors of mice in the three treatment arms collected were stained for CD8⁺ T cells. The cells were counted under a microscope per randomly selected field of view. Study of these PDAC tissues showed that iRGD+GEM therapy expanded CD8⁺ T cells in the tumors (see FIG. 1B and FIG. 1C). iRGD alone did not affect survival but expanded CD8⁺ T cells in the tumor, although to a lesser extent than iRGD+GEM. CD8⁺ T cells in the PDAC of 3 most long-lived and 4 most short-lived KPC mice were analyzed. Tumors from long survivors in the iRGD+GEM arm had particularly high numbers of CD8⁺ T cells (see FIG. 1D). The data suggested that iRGD has an immunomodulatory effect.

### Example 2: Immunomodulation by iRGD

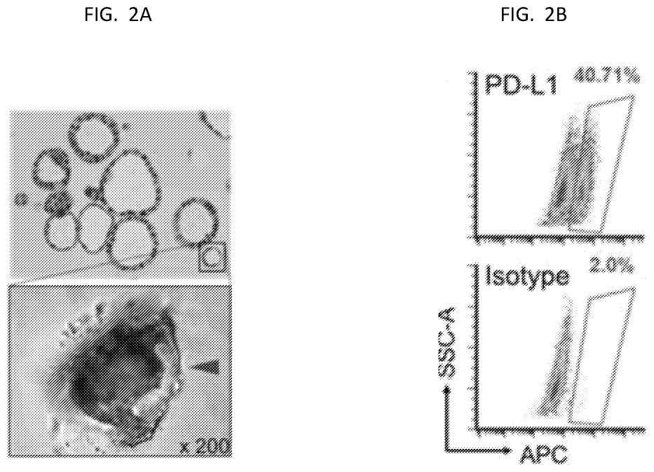

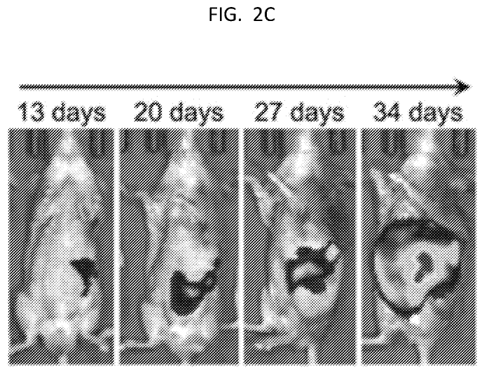

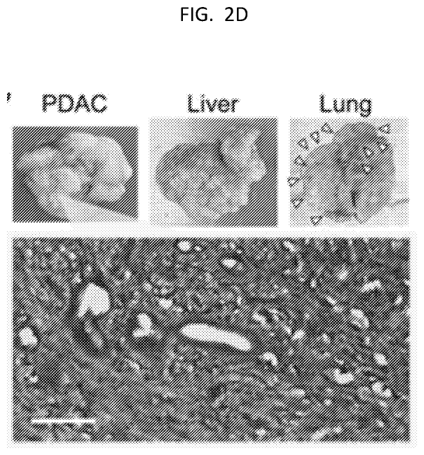

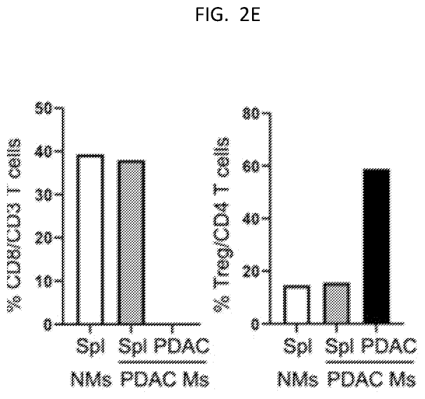

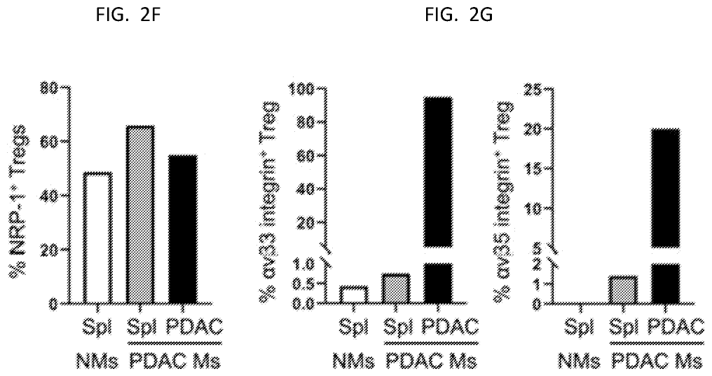

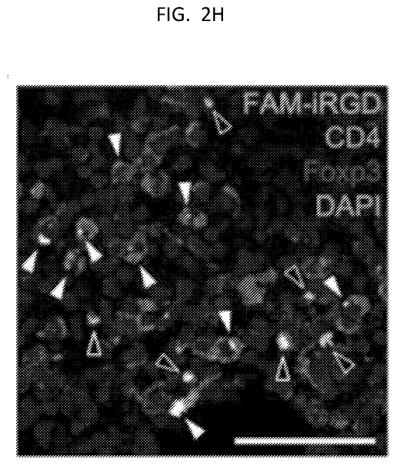

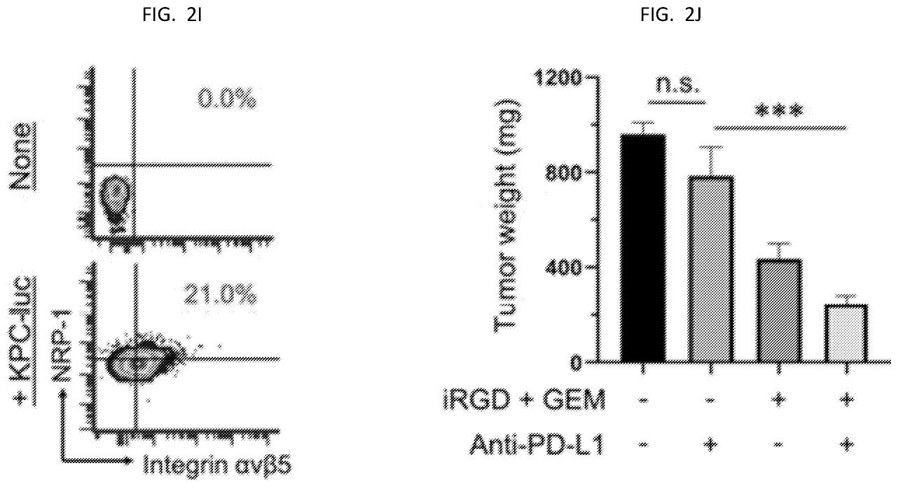

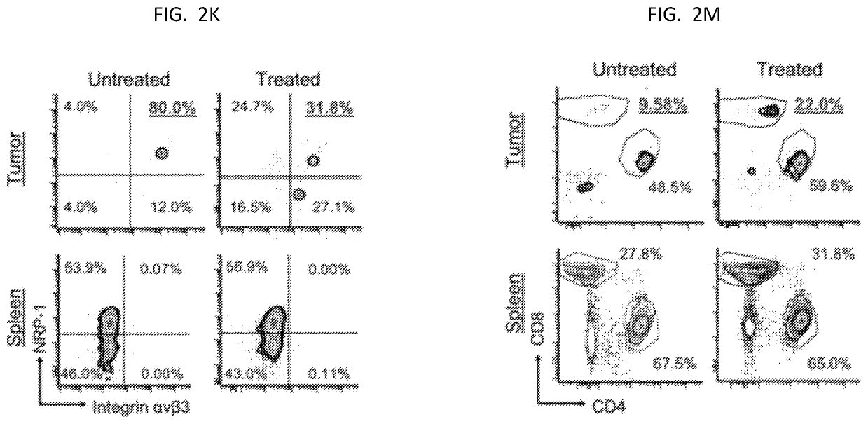

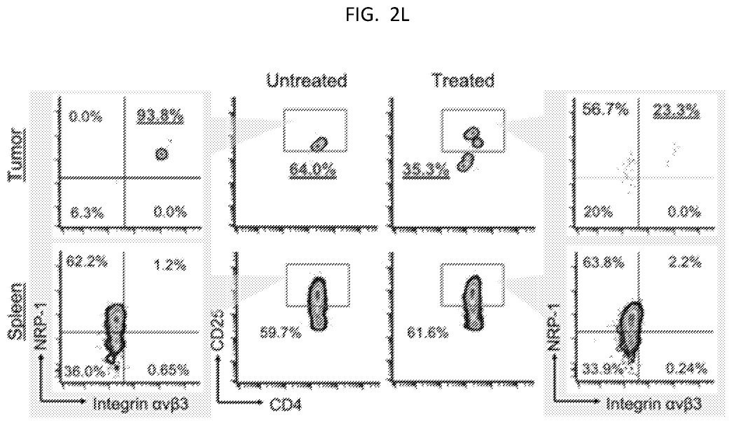

To study the effect of iRGD-based therapies on tumor-infiltrating lymphocytes (TILs) more efficiently, an orthotopic syngeneic PDAC mouse model using organoids established from KPC PDAC tumors was used. FIG. 2A shows that the KPC organoids have elaborate folding and a lumen (arrowhead).

KPC organoids were provided by the Lowy lab at UCSD to the Sugahara lab at Columbia University. Luciferase-positive KPC organoids were prepared and a clone was selected that expressed programmed cell death ligand 1 (PD-L1), an immune checkpoint (KPC-luc) FIG. 2B shows data on PD-L1 expression in KPC-luc organoids analyzed by flow cytometry.

KPC-luc orthotopic tumors grew aggressively. See FIG. 2C, which presents longitudinal luminescence imaging of orthotopic KPC-luc tumors in B6129SF1/J mice. The tumors metastasized to the liver, lung, and peritoneum in about 4 weeks. See FIG. 2D, which shows KPC-luc PDAC and liver and lung metastases. H&E staining of the primary tumor is shown. Scale bar, 100 μm.

The PDAC had rich stromal networks within irregular ductal structures and invasive cancer cells. In the spleen of the KPC-luc mice, 40% of T cells were CD8⁺ and 15% of CD4⁺ T cells were regulatory T cells (Tregs) similar to splenocytes in normal mice. See FIG. 2E. In contrast, (10 day-old) KPC-luc tumors had minimal CD8⁺ T cells but many Tregs (60% of CD4⁺ T cells), in agreement with previous studies in transgenic KPC mice (Clark et al, 2007). About 50-60% of the tumor-infiltrating Tregs expressed NRP-1 similar to splenic Tregs. See FIG. 2F. Surprisingly, >90% of tumor-infiltrating Tregs expressed al/33 integrin and 20% expressed αvβ5 integrin, while only <1-2% of splenic Tregs expressed the integrins. See FIG. 2G. FIGS. 2E, 2F, and 2G show results of flow cytometry of CD8⁺ T cells and Tregs (FIG. 2E), NPR-1⁺ Tregs (FIG. 2F), and αvβ3⁺ and αvβ5⁺ Tregs (FIG. 2G) in PDAC and spleen (Spl) of normal mice (NMs) and KPC-luc mice (PDAC Ms).

Based on the finding that Tregs in PDAC, but not those in the spleen expressed αv integrins and NPR-1 (the receptors of iRGD), studies were performed to determine whether iRGD selectively targets PDAC-infiltrating Tregs. Intravenously injected fluorescein (FAM)-labeled iRGD targeted Tregs in KPC-luc tumors are shown in FIG. 2H. Intravenously injected FAM-iRGD (green) targets CD4⁺ (magenta) Foxp3⁺ (red) Tregs in KPC-luc PDAC (See FIG. 2H, white arrowheads). Some iRGD-targeted Foxp3⁺ cells were CD4^{neg} (black arrowheads). Blue, DAPI; scale bar, 50 μm.

It is likely that the tumor microenvironment had a major contribution because splenic Tregs cultured for 3 days in the presence of KPC-luc cells had increased αv integrins and NRP-1. See FIG. 2I. In this figure, αvβ5 and NRP-1 expression in normal mouse spleen Tregs cultured alone or with KPC-luc cells is shown.

Orthotopic KPC-luc mice were treated with IV iRGD+GEM with or without anti-PD-L1 mAb (clone 10F.9G2) 3× a week for 2 weeks. iRGD+GEM significantly enhanced anti-PD-L1 therapy. See FIG. 2J. In agreement with the depletion of Tregs and expansion of CD8⁺ T cells in the PDAC after iRGD+GEM therapy, a 14-day short-term treatment of orthotopic KPC-luc tumor mice with iRGD+GEM significantly potentiated an immune checkpoint inhibitor, anti-PD-L1 monoclonal antibody (clone 10F.9G2).

The combination led to a significant decrease of both al/33 integrin⁺ NRP-1⁺ (FIG. 2K) and αvβ5 integrin⁺ NRP-1⁺ (not shown) tumor-infiltrating Tregs. NRP-1⁺ splenic Tregs, >99% of which were integrin negative, were not depleted (or even slightly increased). NRP-1⁺ αvβ3 integrin⁺ total Tregs (FIG. 2K) and CD25^{high} Tregs (FIG. 2L, insets), and the proportion of CD8⁺ and CD4⁺ T cells (FIG. 2M), in the PDAC and spleen after iRGD+GEM+anti-PD-L1 mAb treatment. Statistics, ANOVA; n.s., not significant; ***, p<0.001. CD4⁺ CD25^{high} Tregs, which are known to be highly immunosuppressive (Okita et al, 2009; Miyara et al, 2009), were significantly reduced in the treated tumor (see FIG. 2L). About 94% of the CD4⁺ CD25^{high} Tregs were αvβ3 integrin⁺ and NRP-1⁺, which decreased to 23% after the treatment. Tumor-infiltrating CD8⁺ cells doubled with the treatment (see FIG. 2M).

### Example 3: αv Integrin and NRP-1 Expression in Human PDAC Tregs

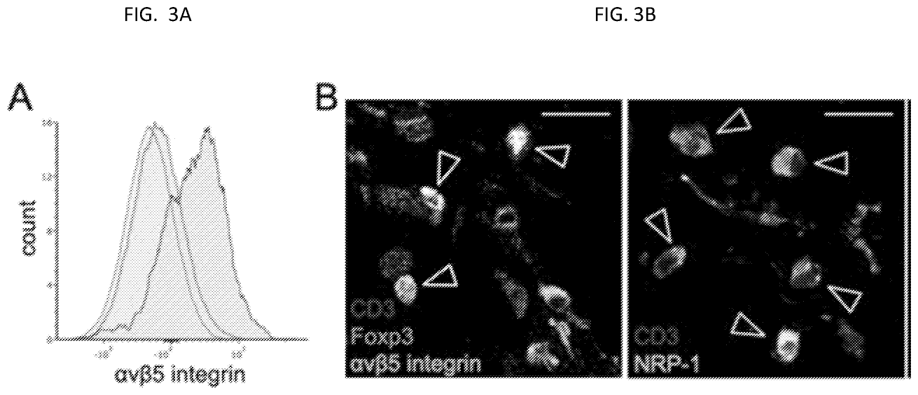

Human PDAC tissue harbors Tregs that express αvβ5 integrins (see FIG. 3A and the left panel of FIG. 3B). NRP-1⁺ T cells were also noted in human PDAC tissue (see FIG. 3B, right panel). The findings suggest the presence of NRP-1⁺ Tregs in PDAC tissue is highly likely in agreement with previous publications that reported the presence of NRP-1⁺ Tregs in human cancer patients. In contrast, Tregs in the spleen of a human PDAC patient or blood from a healthy donor (not shown) did not express αv integrins or NRP-1. Thus, the concept of tumor-specific Treg targeting with iRGD therapy would hold in humans.

FIG. 3 shows αv integrin and NRP-1 expression in human PDAC Tregs. FIG. 3A shows expression of αvβ5 integrin in Tregs isolated from tumor (blue) and spleen (red) samples from a PDAC patient (cell counting data). Green is an isotype control. Scale bars, 20 μm. FIG. 3B shows images of αvβ5 integrin (green) in CD3⁺ (red) Foxp3⁺ (magenta) T cells (white arrowheads) and NRP-1 (green) in CD3⁺ T cells (yellow arrowheads) in human PDAC. Foxp3 was not stained in the right panel due to the incompatibility with NRP-1 staining. DAPI is not shown for better visualization of the other colors. Scale bars, 20 μm.

### Example 4: T Cells in Peritoneal Tumors in Mice Generated with ID8 Mouse Ovarian Cancer Cells

The iRGD peptide sensitizes the cancer to immune checkpoint inhibitors, such as anti-PD-L1, anti-PD-1, and anti-CTLA4 mAbs. iRGD targets Tregs in the tumor and allows effector cells such as CD8⁺ T cells to expand. The effect is further pronounced when iRGD is combined with one or more additional chemotherapeutics such as GEM. The effect is tumor-specific most likely because Tregs that express iRGD receptors are only present in the tumor tissue.

iRGD-based chemotherapy can sensitize various cancer types because cancers other than PDAC, such as ovarian cancer, also harbor αv integrin+ NRP-1+ Tregs (see FIG. 4). Therefore, the findings above can be immediately applied to the clinic in order to solve a major issue in the treatment of various cancers, resistance to immunotherapy.

FIG. 4 relates to T cells in peritoneal tumors in mice generated with ID8 mouse ovarian cancer cells. FIG. 4A shows data on CD8⁺ (4%) and CD4⁺ (17%) T cells; FIG. 4B shows data on CD25^{high} (32%) and CD25^{low} (58%) Tregs; FIG. 4C shows data on αvβ3+ NRP-1⁺ Tregs (63%); FIG. 4D shows data on αvβ5⁺ NRP-1⁺ Tregs (26%). The number of T cells was low since the PTs were small.

### Example 5: Expression of αvβ5 Integrin in Regulatory T Cells

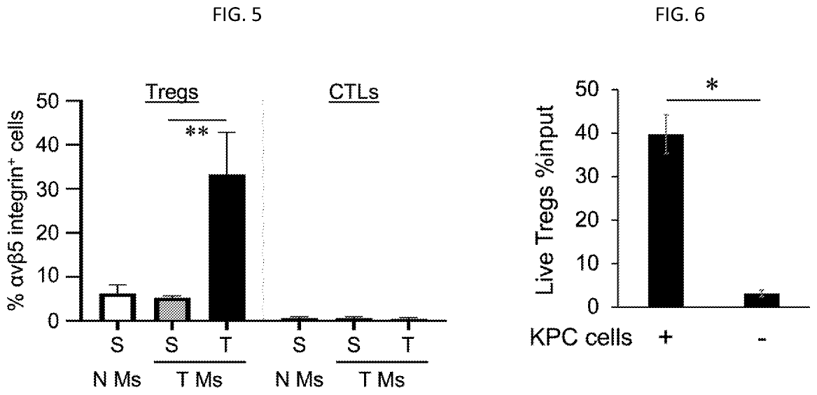

The iRGD peptide itself has an immunomodulatory effect that leads to increased efficacy of checkpoint inhibitors. The expression of αvβ5 integrin is consistently elevated in regulatory T cells (Tregs) that infiltrate pancreatic ductal adenocarcinoma (PDAC) in mice. See FIG. 5, which shows the expression of αvβ5 integrin on Tregs and CTLs isolated from orthotopic PDAC (T) and spleen (S) of KPC-derived syngeneic tumor mice (T Ms) and the spleen of normal mice (N Ms) analyzed by flow cytometry. p **<0.01. These data confirm the findings above.

αvβ5 integrin was not expressed in cytotoxic CD8⁺ T lymphocytes.

Splenic T cells from mice were cultured in the presence of KPC-derived PDAC cells to expand αvβ5 integrin⁺ Tregs. Survival was determined by counting the number of cells using a hemocytometer. *, p<0.01. See results in FIG. 6, which shows the survival of CD4⁺ T cells in the presence or absence of KPC-derived PDAC cells. Interestingly, the cells had a significantly prolonged survival again suggesting that the microenvironment of the PDAC tissue supports the expansion of αvβ5 integrin⁺ Tregs. As stated earlier, co-culturing CD4⁺ T cells with PDAC cells derived from transgenic Kras-LSL^{GD12}, p53-LSL^{172H}, Pdx-1-cre (KPC) mice led to the expansion of αvβ5 integrin⁺ Tregs.

### Example 6

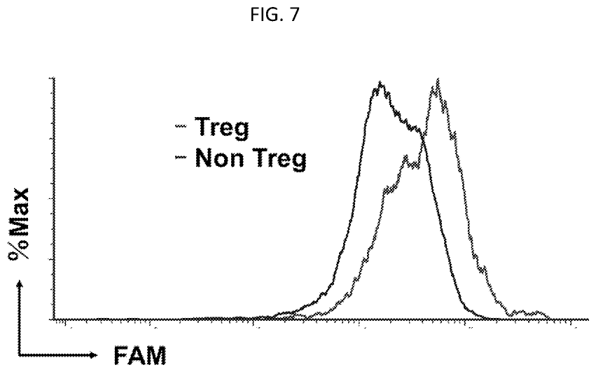

T cells were cultured in the presence of fluorescein (FAM)-labeled iRGD at 37° C. for 1 hour. iRGD binding was determined by flow cytometry. FIG. 7 shows that iRGD binding to CD25⁺ CD4⁺ T cells (Tregs) and CD25^{neg} CD4⁺ T cells (non-Tregs) isolated from KPC-derived PDAC. The data support the earlier finding that iRGD homed to PDAC-infiltrating Tregs in vivo, iRGD effectively bound to Tregs isolated from the mouse PDAC but minimally to CD25^{neg} CD4⁺ T cells (non-Tregs).

### Example 7: iRGD Binding to T Cells

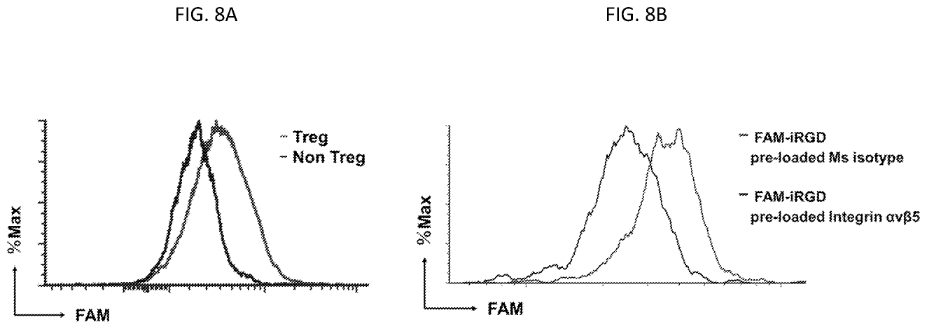

Tregs and non-Tregs were produced by culturing mouse splenic T cells in the presence of CD3/CD28 beads and KPC-derived PDAC cells. FIG. 8 shows data for iRGD binding to CD25⁺ CD4⁺ T cells (Tregs) and CD25^{neg} CD4⁺ T cells (non-Tregs) produced in vitro. FIG. 8A demonstrates that FAM-iRGD binding to the Tregs was determined by flow cytometry. FIG. 8B shows that anti-αvβ5 integrin Abs inhibited FAM-iRGD binding to the Tregs. iRGD also bound to cultured Tregs (but minimally to non-Tregs), which were expanded in the condition that induces αvβ5 integrin expression. The iRGD binding to the Tregs was inhibited by anti-αvβ5 integrin antibodies (Abs) confirming receptor-mediated binding of iRGD.

### Example 8: Effect of iRGD Monotherapy on Tregs and the CTL/Treg Ratio in the PDAC Tissue and Spleen

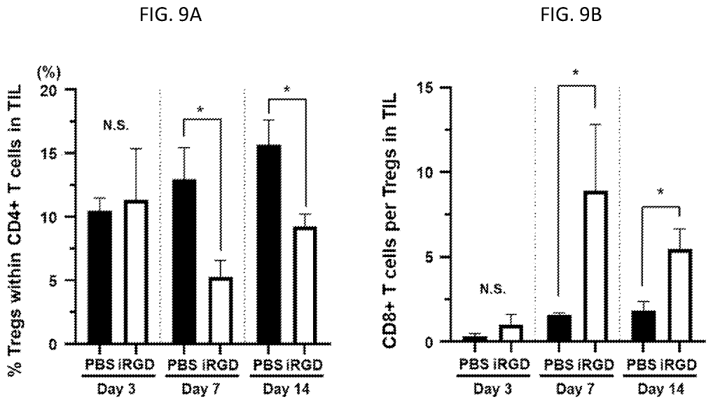

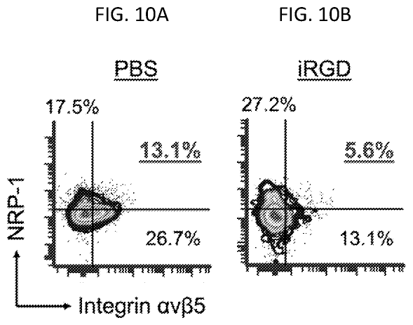

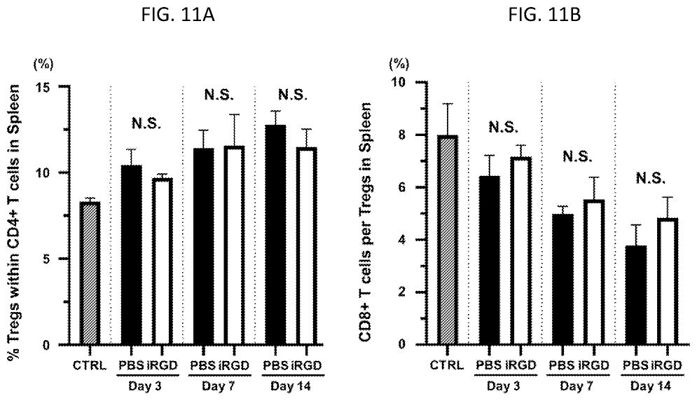

Mice bearing orthotopic PDAC were treated with systemic iRGD or PBS for 2 weeks. See results in FIG. 9, FIG. 10, and FIG. 11, which show the effect of iRGD monotherapy on Tregs and the CTL/Treg ratio in the PDAC tissue and spleen. FIG. 9A and FIG. 9B show time-dependent changes in the proportion of Tregs and CTL/Treg ratio in the PDAC tissue. Systemic treatment of the PDAC mice with iRGD monotherapy significantly decreased PDAC-infiltrating Tregs and increased the CTL/Treg ratio.

FIG. 10A and FIG. 10B show results for αvβ5 integrin⁺ and NRP-1⁺ Tregs in the PDAC after iRGD monotherapy. There was a significant decrease of αvβ5 integrin⁺ Tregs in the PDAC tissue. FIG. 11A and FIG. 11B shows time-dependent changes in the proportion of Tregs and CTL/Treg ratio in the spleen. *, p<0.05; n.s., not significant. There was no change in the Tregs or the CTL/Treg ratio in the spleen, supporting our finding that there are minimal αvβ5 integrin⁺ Tregs in the spleen. These results suggest that iRGD helps restore anti-cancer T cell immunity.

### Example 9: iRGD Effects on Anti-Tumor Efficacy

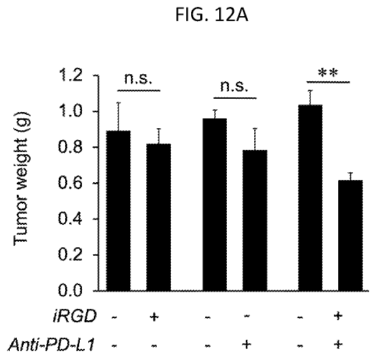

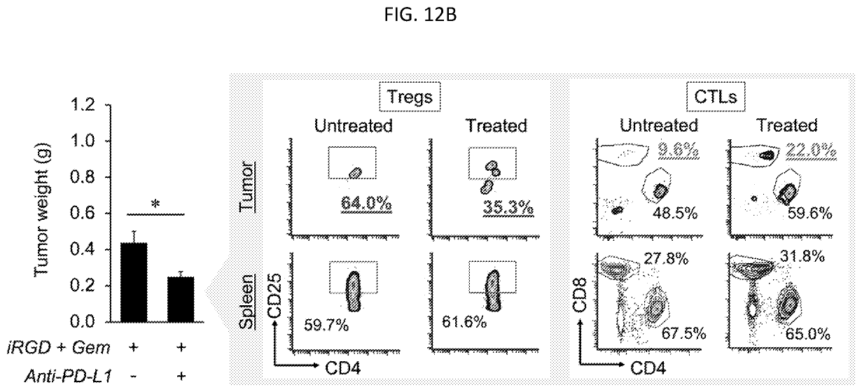

KPC-derived PDAC mice were treated with iRGD±anti-PD-L1 mAb (A; n=4-6) or iRGD+Gem±anti-PD-L1 mAb (B; n=4) 3× a week for 2 weeks. Flow cytometry data of CD4⁺ CD25⁺ Tregs and CD8⁺ T cells in the tumor and spleen after iRGD+Gem+anti-PD-L1 mAb therapy are shown in FIG. 12. Tregs halved and CTLs doubled in the PDAC but not in the spleen. n.s., not significant; *, p<0.05; **, p<0.01. iRGD significantly enhanced the anti-tumor efficacy of an anti-programmed cell death ligand 1 (PD-L1) Ab in PDAC mice, while iRGD alone or anti-PD-L1 Ab alone had no effect (FIG. 12A). Adding gemcitabine further enhanced the efficacy of the iRGD+anti-PD-L1 therapy in the PDAC mice (FIG. 12B).

### REFERENCES

All references listed below and throughout the specification are hereby incorporated by reference in their entirety.
1. U.S. Pat. No. 9,115,170, Jan. 30, 2013.
2. U.S. Pat. No. 10,669,311, Apr. 22, 2016.
3. United States Patent Publication No. 2018-0236076, Aug. 2, 2016.
4. United States Patent Publication No. 2018-0319886, Nov. 8, 2018.
5. United States Patent Publication No. 2017-0246298, Aug. 31, 2017.
6. United States Patent Publication No. 2018-0221362, Jul. 28, 2016.
7. U.S. Pat. No. 10,604,526, Nov. 19, 2018.
8. International Patent Publication No. WO/2018/137658, Jan. 24, 2018.
9. United States Patent Publication No. 2020-0156935, Jul. 29, 2019.
10. U.S. Pat. No. 10,300,143, Oct. 2, 2017.
11. Sharma P, Hu-Lieskovan S, Wargo J A, Ribas A. Primary, adaptive, and acquired resistance to cancer immunotherapy. Cell. 2017 Feb. 9; 168(4): pp. 707-723.
12. Clark C E, Hingorani S R, Mick R, Combs C, Tuveson D A, Vonderheide R H. Dynamics of the immune reaction to pancreatic cancer from inception to invasion. Cancer Res. 2007 Oct. 1; 67(19): pp. 9518-27.
13. Brahmer J R, Lacchetti C, Schneider B J, Atkins M B, Brassil K J, Caterino J M, Chau I, Ernstoff M S, Gardner J M, Ginex P, Hallmeyer S, Holter Chakrabarty J, Leighl N B, Mammen J S, McDermott D F, Naing A, Nastoupil U, Phillips T, Porter L D, Puzanov I, Reichner C A, Santomasso B D, Seigel C, Spira A, Suarez-Almazor M E, Wang Y, Weber J S, Wolchok J D, Thompson J A; National Comprehensive Cancer Network. Management of immune-related adverse events in patients treated with immune checkpoint inhibitor therapy: American Society of Clinical Oncology Clinical Practice Guideline. J Clin Oncol. 2018 Jun. 10; 36(17): pp. 1714-1768.
14. Sugahara K N, Teesalu T, Karmali P P, Kotamraju V R, Agemy L, Greenwald D R, Ruoslahti E. Coadministration of a tumor-penetrating peptide enhances the efficacy of cancer drugs. Science. 2010 May 21; 328(5981): pp. 1031-5.
15. Cancer Cell, 16(6): 510-520, 2009.
16. Cell Rep., 23(11): 3262-3274, 2018.
17. Sci. Trans. Med., 8(352): 2016.
18. Mol. Cancer Ther., 17(11): 2377-2388, 2018.
19. Sci. Rep., 8(1): article 2274, 2018.
20. Nat. Commun., 10(1): article 1336, 2019.
21. J. Control Release, 281: 84-96, 2018.
22. Ann. Oncol., 31(suppl. 4), 2020.
23. Balachandran V P, Beatty G L, Dougan S K. Broadening the impact of immunotherapy to pancreatic cancer: Challenges and opportunities. Gastroenterology, 156:2056-72, 2019. PMCID: PMC6486864.
24. Brahmer J R, Lacchetti C, Schneider B J, Atkins M B, Brassil K J, Caterino J M, Chau I, Ernstoff M S, Gardner J M, Ginex P, Hallmeyer S, Holter Chakrabarty J, Leighl N B, Mammen J S, McDermott D F, Naing A, Nastoupil U, Phillips T, Porter L D, Puzanov I, Reichner C A, Santomasso B D, Seigel C, Spira A, Suarez-Almazor M E, Wang Y, Weber J S, Wolchok J D, Thompson J A; National Comprehensive Cancer Network. Management of Immune-Related Adverse Events in Patients Treated With Immune Checkpoint Inhibitor Therapy: American Society of Clinical Oncology Clinical Practice Guideline. J Clin Oncol, 36:1714-68, 2018. PMCID: PMC6481621.
25. Chaudhary B, Khaled Y S, Ammori B J, Elkord E. Neuropilin 1: function and therapeutic potential in cancer. Cancer Immunol Immunother, 63:81-99, 2014. PMID: 24263240.
26. Clark C E, Hingorani S R, Mick R, Combs C, Tuveson D A, Vonderheide R H. Dynamics of the immune reaction to pancreatic cancer from inception to invasion. Cancer Res, 67:9518-27, 2007. PMID: 17909062.
27. Dean A, Gill S, McGregor M, Broadbridge V, Jarvilainen H A, Price T. Phase I trial of the first-in-class agent CEND-1 in combination with gemcitabine and nab paclitaxel in patients with metastatic pancreatic cancer. Presented at: ESMO Virtual Congress 2020, Abstract 1528P.
28. Miyara M, Yoshioka Y, Kitoh A, Shima T, Wing K, Niwa A, Parizot C, Taflin C, Heike T, Valeyre D, Mathian A, Nakahata T, Yamaguchi T, Nomura T, Ono M, Amoura Z, Gorochov G, Sakaguchi S. Functional delineation and differentiation dynamics of human CD4+ T cells expressing the FoxP3 transcription factor. Immunity, 30:899-911, 2009. PMID: 19464196.
29. Okita R, Yamaguchi Y, Ohara M, Hironaka K, Okawaki M, Nagamine I, Ikeda T, Emi A, Hihara J, Okada M. Targeting of CD4+CD25^{high} cells while preserving CD4+CD25low cells with low-dose chimeric anti-CD25 Ab in adoptive immunotherapy of cancer. Int J Oncol, 34:563-72, 2009. PMID: 19148493.
30. Pang H B, Braun G B, Friman T, Aza-Blanc P, Ruidiaz M, Sugahara K N, Teesalu T, Ruoslahti E. An endocytosis pathway initiated through neuropilin-1 and regulated by nutrient availability. Nat Commun, 5:4904, 2014. PMCID: PMC4185402.
31. Roy S, Bag A K, Singh R K, Talmadge J E, Batra S K, Datta K. Multifaceted Role of Neuropilins in the Immune System: Potential Targets for Immunotherapy. Front Immunol, 8:1228, 2017. PMCID: PMC5641316.
32. Sugahara K N, Teesalu T, Karmali P P, Kotamraju V R, Agemy L, Girard O M, Hanahan D, Mattrey R F, Ruoslahti E. Tissue-penetrating delivery of compounds and nanoparticles into tumors. Cancer Cell, 16:510-20, 2009. PMCID: PMC2791543.
33. Sugahara K N, Scodeller P, Braun G B, de Mendoza T H, Yamazaki C M, Kluger M D, Kitayama J, Alvarez E, Howell S B, Teesalu T, Ruoslahti E, Lowy A M. A tumor-penetrating peptide enhances circulation-independent targeting of peritoneal carcinomatosis. J Control Release, 212:59-69, 2015a. PMCID: PMC4508207.
34. Von Hoff D D, Ervin T, Arena F P, Chiorean E G, Infante J, Moore M, Seay T, Tjulandin S A, Ma W W, Saleh M N, Harris M, Reni M, Dowden S, Laheru D, Bahary N, Ramanathan R K, Tabernero J, Hidalgo M, Goldstein D, Van Cutsem E, Wei X, Iglesias J, Renschler M F. Increased survival in pancreatic cancer with nab-paclitaxel plus gemcitabine. N Engl J Med, 369:1691-703, 2013. PMCID: PMC4631139.
35. Ding et al., Nat. Commun. 10:1336, 2019.

1. A method of treating cancer in a subject, comprising: administering to the subject an iRGD peptide, or peptide variant thereof, or iRGD conjugate in combination with one or more immune checkpoint inhibitor.

2. The method of claim 1, wherein the iRGD peptide comprises SEQ ID NO:3.

3. The method of claim 1, wherein the immune checkpoint inhibitor comprises a PD-1 inhibitor, a PD-L1 inhibitor, or a PD-L2 inhibitor or any combination thereof.

4. The method of claim 3, wherein the one or more immune checkpoint inhibitor comprises 2, 3, or 4 immune checkpoint inhibitors.

5. The method of claim 3, wherein the immune checkpoint inhibitor is selected from the group consisting of ipilimumab, tremilimumab, nivolumab, pembrolizumab (lambrolizumab), pidilizumab, MPDL3280A, BMS-936559, MPDL3280A, MEDI4736, MSB0010718C, or any combination thereof.

6. The method of claim 5, wherein the immune checkpoint inhibitor is selected from the group consisting of ipilimumab, tremilimumab, pembrolizumab, pidilizumab, and any combination thereof.

7. The method of claim 1, wherein the cancer is a metastatic cancer.

8. The method of claim 1, wherein the cancer is immunotherapy-refractory.

9. The method of claim 1, wherein the cancer expresses one or more inhibitory immune checkpoint molecule.

10. The method of claim 1, wherein the cancer is unresectable.

11. The method of claim 1, wherein the cancer is stage I, stage II, stage III, or stage IV cancer.

12. The method of claim 1, wherein the cancer is selected from the group consisting of pancreatic duct adenocarcinoma, malignant melanoma, ovarian cancer, brain, breast, lung, liver, bile duct, GI tract, prostate, uterine cancers, mesothelioma, sarcoma.

13. The method of claim 1, wherein the cancer is pancreatic duct adenocarcinoma.

14. The method of claim 1, further comprising administering to the subject an adjunct cancer therapy selected from the group consisting of surgery, radiation therapy, additional immunotherapy, and chemotherapy.

15. The method of claim 14, wherein the chemotherapy is treatment with an adjunct cancer agent.

16. The method of claim 1, wherein the method sensitizes a cancer to immune checkpoint inhibitor immunotherapy.
